(19) United States
(12) Patent Application Publication
(10) Pub. No.: US 2026/0325481 A1
(43) Pub. Date: Oct. 22, 2026

(54) THERAPY SELECTION AND TREATMENT OF NEURODEGENERATIVE DISORDERS

(71) Applicant: Anavex Life Sciences Corp., New York, NY (US)

(72) Inventor: Christopher U. Missling, New York, NY (US)

(21) Appl. No.: 19/099,422

(22) PCT Filed: Jul. 31, 2023
(86) PCT No.: PCT/US2023/071350

§ 371 Date: Jan. 28, 2025

Related U.S. Application Data
(60) Provisional application No. 63/393,573, filed on Jul. 29, 2022.
(60) Provisional application No. 63/523,281, filed on Jun. 26, 2023.

(51) Int. Cl. A61K 31/341; A61P 25/16; A61P 25/28; C12Q 1/6883

(52) U.S. Cl. CPC A61K 31/341; A61P 25/16; A61P 25/28; C12Q 1/6883; C12Q 2600/158

(57) ABSTRACT

The present disclosure provides methods for individualized selection of, and monitoring of treatment using a neurodegenerative therapeutic agent such as Sigma-1 receptor agonist therapy for a subject, based on changes in gene expression pre- and post-treatment. The present disclosure also provides methods to evaluate current status or risk of a subject in developing neurodegenerative disorders. Also provided are kits for practicing the methods.

### CROSS REFERENCE TO RELATED APPLICATIONS

This application claims the benefit of U.S. Provisional Patent Application No. 63/393,573 filed Jul. 29, 2022, and U.S. Provisional Patent Application No. 63/523,281 filed Jun. 26, 2023, both of which are entitled “THERAPY SELECTION AND TREATMENT OF NEURODEGENERATIVE DISORDERS”, and the entire disclosure of both of which is incorporated herein by reference.

### FIELD OF THE INVENTION

The present disclosure relates generally to personalized medical treatments and more specifically to genetic methods and kits for individualized monitoring of medical treatments and selection of therapeutic agents.

### BACKGROUND OF THE INVENTION

Genomics is an interdisciplinary field of biology focusing on the structure, function, evolution, mapping, and editing of genomes. Over the last few decades, genomic analysis and profiling have become tools useful to diagnose, treat, and prevent diseases. But there are few reports on how to use genomic profiling to assess the effectiveness of a therapy or selecting a proper therapeutic agent for a specific patient.

### SUMMARY OF THE INVENTION

One aspect of the present disclosure encompasses a method of selecting a therapeutic agent for a subject suffering from a disease or a disorder selected from Alzheimer's disease, Parkinson's disease, Huntington's disease, Amyotrophic lateral sclerosis, Prion disease, diabetic cardiomyopathy, non-alcoholic fatty liver disease, a coronavirus disease, a disorder related to thermogenesis, a disorder related to oxidative phosphorylation, a disorder related to proteasome dysfunction, a disorder related to ribosome dysfunction, a disorder related to retrograde endocannabinoid signaling, and a disorder related to cardiac muscle contraction. The method comprises obtaining or having obtained a first biological sample from the subject; administering to the subject a Sigma receptor agonist for a period of time; obtaining or having obtained a second biological sample of the subject at the end of administration period; determining a gene expression profile for the first biological sample and for the second biological sample; comparing the gene expression profile of the first biological sample with that of the second biological sample to identify at least one differentially expressed gene and/or overrepresented gene cluster; and selecting the Sigma receptor agonist as the therapeutic agent for the subject, if one of the following conditions is observed: (i) at least one differentially expressed gene is a gene listed in a first gene cluster comprising MS4A4A, COX5B, COX7B, COX8A, NDUFB6, TXN, PSMA4, CLTB, PSMB3, POLR2F, NDUFA1, PSMD13, POLR2, NDUFA2, DDIT3, CYBA, AGER, MAPA2K2, PSENEN, or NDUFB3; or a gene listed in a second gene cluster comprising COX4I1, NDUFB11, PSMA7, COX6A1, NDUFB2, PSMB1, COX6B1, NDUFB7, PSMB4, COX6C, NDUFB8, PSMB5, COX7A2, NDUFB9, PSMB6, COX7C, NDUFC1, PSMB7, CYC1, NDUFC2, PSMD8, NDUFA11, NDUFS3, RPS27A, NDUFA12, NDUFS4, SLC25A6, NDUFA13, NDUFS5, TXN2, NDUFA3, NDUFS6, UQCR10, NDUFA4, NDUFS7, UQCR11, NDUFA7, NDUFS8, UQCRH, NDUFA8, PARK7, UQCRQ, NDUFB1, PSMA2, NDUFB10, CDK5, GAPDH, HRAS, HSD17B10, or PSMA6; or (ii) the identified overrepresented gene cluster(s) overlaps with genes listed in the first gene cluster or in the second gene cluster. In any of the methods, the therapeutic agent can comprise a Sigma receptor agonist. The Sigma receptor agonist can comprise ANAVEX2-73 (A2-73), A2-73 free base, ANAVEX 19-144, ANAVEX1-41, AV1066, ANAVEX3-71, PRE-084, Donepezil, Fluvoxamine, Amitriptyline, L-687,384, SA-4503, Dextromethorphan, Dimethyltryptamine, (+)-pentazocine, any of their crystal forms, enantiomers and pharmaceutically acceptable salts thereof, or any combinations thereof. In any of the methods, the therapeutic agent can comprise a Sigma receptor agonist selected from ANAVEX2-73 (A2-73), A2-73 free base, A2-73 amorphous form, A2-73 Crystal Form I, A2-73 Crystal Form II, A2-73 Crystal Form III, (−) A2-73 enantiomer, or (+) A2-73 enantiomer, or a combination thereof.

In the methods, the therapeutic agent can be administered for a period up to 14 weeks, such as about 4 weeks, about 5 weeks, about 6 weeks, about 7 weeks, about 8 weeks, about 9 weeks, about 10 weeks, about 11 weeks, about 12 weeks, about 13 weeks, or about 14 weeks. And the therapeutic agent is administered daily, such as once daily.

In various other aspects of the methods, the Sigma receptor agonist can comprise A2-73, A2-73 free base, or any combination thereof. In another aspect of the methods, the Sigma receptor agonist can comprise A2-73 in crystal form, and is administered in the amount of about 40 mg to about 60 mg once daily for about 6-11 weeks. For example, A2-73 in a crystal form is administered at a dose of about 50 mg daily for up to 11 weeks. Alternatively, A2-73 is administered daily in an escalating dose starting from about 10 mg to ending at about 50 mg once daily for 6-11 weeks. In another aspect, the Sigma receptor agonist comprises A2-73, A2-73 free base, or a combination thereof. In one aspect, A2-73 or A2-73 free base is administered daily in an amount of about 40-60 mg for a period of 6 to 14 weeks. In yet another aspect, the Sigma receptor agonist comprises A2-73 or A2-73 free base in a crystal form, and wherein the A2-73 or A2-73 free base is administered once daily in an amount of about 50 mg for a period of up to 11 weeks. In yet another aspect, the therapeutic agent is a Sigma receptor agonist, and the Sigma receptor agonist comprises A2-73 in a crystal form. The A2-73 crystal is administered once daily for a period of 11 weeks in an escalating amount starting at about 10 mg and ending at about 50 mg.

Another aspect of the present disclosure encompasses a method of evaluating effectiveness of a neurodegenerative therapy for a subject in need thereof. The method comprises obtaining or having obtained a first biological sample from the subject; administering to the subject the neurodegenerative therapy for a period of time; obtaining or having obtained a second biological sample of the subject at the end of therapy period; determining a gene expression profile for the first biological sample and for the second biological sample; comparing the gene expression profile of the first biological sample with that of the second biological sample to identify differentially expressed genes and overrepresented gene clusters; and identifying the neurodegenerative therapy as effective if one of the following conditions is met: (i) an identified differentially expressed gene is a gene listed in a first gene cluster comprising MS4A4A, COX5B, COX7B, COX8A, NDUFB6, TXN, PSMA4, CLTB, PSMB3, POLR2F, NDUFA1, PSMD13, POLR2, NDUFA2, DDIT3, CYBA, AGER, MAPA2K2, PSENEN, or NDUFB3; or (ii) an identified differentially expressed gene is a gene listed in a second gene cluster comprising COX4I1, NDUFB11, PSMA7, COX6A1, NDUFB2, PSMB1, COX6B1, NDUFB7, PSMB4, COX6C, NDUFB8, PSMB5, COX7A2, NDUFB9, PSMB6, COX7C, NDUFC1, PSMB7, CYC1, NDUFC2, PSMD8, NDUFA11, NDUFS3, RPS27A, NDUFA12, NDUFS4, SLC25A6, NDUFA13, NDUFS5, TXN2, NDUFA3, NDUFS6, UQCR10, NDUFA4, NDUFS7, UQCR11, NDUFA7, NDUFS8, UQCRH, NDUFA8, PARK7, UQCRQ, NDUFB1, PSMA2, NDUFB10, CDK5, GAPDH, HRAS, HSD17B10, or PSMA6; or (iii) an identified overrepresented gene cluster(s) overlaps with genes listed in the first gene cluster or in the second gene cluster, wherein the neurodegenerative therapy is identified as ineffective for the subject if none of the foregoing conditions are met.

In another aspect of the methods, if the neurodegenerative therapy is identified as ineffective for a subject, a follow-up action can be taken, such as switching to another neurodegenerative therapy, supplementing with another neurodegenerative therapy, adjusting dose if the neurodegenerative therapy being a drug therapy, or switching to a combined neurodegenerative therapy.

In any of the methods, the subject may be a human subject having or suspected of having a neurodegenerative disease, such as Alzheimer's disease, Parkinson's disease, Huntington's disease, Amyotrophic lateral sclerosis, Prion disease, or any combination thereof.

In any of the methods, the neurodegenerative therapy can be a drug therapy comprising ANAVEX2-73 (A2-73), ANAVEX 19-144, ANAVEX1-41, AV1066, ANAVEX3-71, PRE-084, Donepezil, Fluvoxamine, Amitriptyline, L-687,384, SA-4503, Dextromethorphan, Dimethyltryptamine, (+)-pentazocine, or any of their crystal forms, enantiomers and pharmaceutically acceptable salts thereof. For example, the neurodegenerative therapy is a A2-73 drug therapy comprising A2-73 free base, A2-73 amorphous form, A2-73 Crystal Form I, A2-73 Crystal Form II, A2-73 Crystal Form III, (−) A2-73 enantiomer, or (+) A2-73 enantiomer.

In yet another aspect, the neurodegenerative therapy can be administered for a period up to 14 weeks, such as about 4 weeks, about 5 weeks, about 6 weeks, about 7 weeks, about 8 weeks, about 9 weeks, about 10 weeks, about 11 weeks, about 12 weeks, about 13 weeks, or about 14 weeks. The therapeutic agent may be administered daily, such as once or twice daily.

In yet another aspect of the methods, the neurodegenerative therapy is a drug therapy comprising A2-73, for example in a crystal form, and is administered in the amount of about 40 mg to about 60 mg once daily for about 6-11 weeks. For example, A2-73 is administered in about 50 mg daily for up to 11 weeks. Alternatively, the A2-73 is administered daily in an escalating dose starting from about 10 mg to ending at about 50 mg once daily for 6-11 weeks.

Yet another aspect of the present disclosure encompasses a method of identifying a subject responsive to a Sigma-1 receptor agonist therapy. The method comprises obtaining or having obtained a first biological sample from the subject; administering to the subject a Sigma receptor agonist for a period of time; obtaining or having obtained a second biological sample of the subject at the end of administration period; determining a gene expression profile for the first biological sample and for the second biological sample; comparing the gene expression profile of the first biological sample with that of the second biological sample, to identify differentially expressed genes and overrepresented gene clusters; and identifying the subject as responsive to the Sigma-1 receptor agonist therapy, if any one of the following conditions is observed: (i) the identified differentially expressed gene is a gene listed in a first gene cluster comprising MS4A4A, COX5B, COX7B, COX8A, NDUFB6, TXN, PSMA4, CLTB, PSMB3, POLR2F, NDUFA1, PSMD13, POLR2, NDUFA2, DDIT3, CYBA, AGER, MAPA2K2, PSENEN, or NDUFB3; or a gene listed in a second gene cluster comprising COX4I1, NDUFB11, PSMA7, COX6A1, NDUFB2, PSMB1, COX6B1, NDUFB7, PSMB4, COX6C, NDUFB8, PSMB5, COX7A2, NDUFB9, PSMB6, COX7C, NDUFC1, PSMB7, CYC1, NDUFC2, PSMD8, NDUFA11, NDUFS3, RPS27A, NDUFA12, NDUFS4, SLC25A6, NDUFA13, NDUFS5, TXN2, NDUFA3, NDUFS6, UQCR10, NDUFA4, NDUFS7, UQCR11, NDUFA7, NDUFS8, UQCRH, NDUFA8, PARK7, UQCRQ, NDUFB1, PSMA2, NDUFB10, CDK5, GAPDH, HRAS, HSD17B10, or PSMA6; or (ii) the identified overrepresented gene cluster(s) overlaps with genes listed in the first gene cluster or in the second gene cluster.

In any of the methods, the subject being administered the Sigma-1 receptor agonist therapy is a human subject having or being suspected of having a disorder or disease, such as Alzheimer's disease, Parkinson's disease, Huntington's disease, Amyotrophic lateral sclerosis, Prion disease, diabetic cardiomyopathy, non-alcoholic fatty liver disease, a coronavirus disease, a disorder related to thermogenesis, a disorder related to oxidative phosphorylation, a disorder related to proteasome dysfunction, a disorder related to ribosome dysfunction, a disorder related to retrograde endocannabinoid signaling, or a disorder related to cardiac muscle contraction.

In various aspects of the methods, the Sigma-1 receptor agonist therapy may comprise ANAVEX2-73 (A2-73), A2-73 free base, ANAVEX 19-144, ANAVEX1-41, AV1066, ANAVEX3-71, PRE-084, Donepezil, Fluvoxamine, Amitriptyline, L-687,384, SA-4503, Dextromethorphan, Dimethyltryptamine, (+)-pentazocine, or any of their crystal forms, enantiomers and pharmaceutically acceptable salts thereof. For example, the Sigma-1 receptor agonist therapy is a A2-73 drug therapy comprising A2-73 free base, A2-73 amorphous form, A2-73 Crystal Form I, A2-73 Crystal Form II, A2-73 Crystal Form III, (−) A2-73 enantiomer, or (+) A2-73 enantiomer.

In various aspects of the methods, the Sigma-1 receptor agonist therapy is administered for a period up to 14 weeks, such as about 4 weeks, about 5 weeks, about 6 weeks, about 7 weeks, about 8 weeks, about 9 weeks, about 10 weeks, about 11 weeks, about 12 weeks, about 13 weeks, or about 14 weeks. And the therapeutic agent is administered daily, such as once daily.

In various aspects of the methods, the Sigma-1 receptor agonist therapy can comprise a crystal form of A2-73 or A2-73 free base, and is administered in the amount of about 40 mg to about 60 mg once daily for about 6-11 weeks. For example, a A2-73 crystal or a A2-73 free base crystal is administered in about 50 mg daily for up to 11 weeks. Or A2-73 or A2-73 free base is administered daily in an escalating dose starting from about 10 mg to ending at about 50 mg once daily for 6-11 weeks.

Another aspect of the present disclosure encompasses a method of determining if a subject is having or is suspected of having a disease or disorder, or assessing a risk of a subject for developing such a disease or disorder selected from Alzheimer's disease, Parkinson's disease, Parkinson's disease with dementia, Huntington's disease, Amyotrophic lateral sclerosis, Prion disease, diabetic cardiomyopathy, non-alcoholic fatty liver disease, a coronavirus disease, a disorder related to thermogenesis, a disorder related to oxidative phosphorylation, a disorder related to proteasome dysfunction, a disorder related to ribosome dysfunction, a disorder related to retrograde endocannabinoid signaling, or a disorder related to cardiac muscle contraction. The method comprises obtaining or having obtained a first biological sample from the subject; administering to the subject a composition of ANAVEX 2-73 for a period of time; obtaining or having obtained a second biological sample of the subject at the end of administration period; determining a gene expression profile for the first biological sample and for the second biological sample; comparing the gene expression profile of the first biological sample with that of the second biological sample to identify differentially expressed genes and overrepresented gene clusters; and identifying the subject as having a disorder or disease as listed above, or as having increased risk of developing such a disorder or disease, if any one of the following conditions is observed: (i) the identified differentially expressed gene is a gene listed in a first gene cluster comprising MS4A4A, COX5B, COX7B, COX8A, NDUFB6, TXN, PSMA4, CLTB, PSMB3, POLR2F, NDUFA1, PSMD13, POLR2, NDUFA2, DDIT3, CYBA, AGER, MAPA2K2, PSENEN, or NDUFB3; or a gene listed in a second gene cluster comprising COX4I1, NDUFB11, PSMA7, COX6A1, NDUFB2, PSMB1, COX6B1, NDUFB7, PSMB4, COX6C, NDUFB8, PSMB5, COX7A2, NDUFB9, PSMB6, COX7C, NDUFC1, PSMB7, CYC1, NDUFC2, PSMD8, NDUFA11, NDUFS3, RPS27A, NDUFA12, NDUFS4, SLC25A6, NDUFA13, NDUFS5, TXN2, NDUFA3, NDUFS6, UQCR10, NDUFA4, NDUFS7, UQCR11, NDUFA7, NDUFS8, UQCRH, NDUFA8, PARK7, UQCRQ, NDUFB1, PSMA2, NDUFB10, CDK5, GAPDH, HRAS, HSD17B10, or PSMA6; or (ii) the identified overrepresented gene cluster(s) overlaps with genes listed in the first gene cluster or in the second gene cluster. In various aspects of the method, the disease is Parkinson's disease, Parkinson's disease with dementia, or Prion disease. The A2-73 composition may comprise A2-73 free base, A2-73 amorphous form, A2-73 Crystal Form I, A2-73 Crystal Form II, A2-73 Crystal Form III, (−) A2-73 enantiomer, (+) A2-73 enantiomer, or any combination thereof. The A2-73 composition may be administered for a period up to 14 weeks, such as about 4 weeks, about 5 weeks, about 6 weeks, about 7 weeks, about 8 weeks, about 9 weeks, about 10 weeks, about 11 weeks, about 12 weeks, about 13 weeks, or about 14 weeks. The A2-73 composition may be administered daily, such as once daily. The A2-73 composition may comprise a crystal of A2-73 or A2-73 free base, and may be is administered in the amount of about 40 mg to about 60 mg once daily for about 6-11 weeks. For example, A2-73 is administered in about 50 mg daily for up to 11 weeks. Or A2-73 is administered daily in an escalating dose starting from about 10 mg to ending at about 50 mg once daily for 6-11 weeks.

In any of the methods, the first and second biological samples can each be selected from a blood serum sample, a blood plasma sample, or any biological sample suitable for transcriptomic analysis as disclosed herein.

Another aspect of the present disclosure encompasses a kit for a medical use. The kit includes several containers, for example a first container to receive a first biological sample; a second container to receive a second biological sample; a third and any additional containers useful for storing any reagents or components needed for sequencing or measuring gene expression; and a container useful for storing therapeutic compounds or compositions as described herein, such as any Sigma-1 receptor agonist, for example an A2-73 composition. The kit may also include instructions in printed or computer readable medium describing the methods disclosed herein and use of the kit to perform the methods.

For example, a kit as disclosed herein may be used for any of the various disclosed methods, such as selection of a therapeutic agent for a subject, evaluation of the effectiveness of a neurodegenerative therapy, identification of a subject responsive to a Sigma-1 receptor agonist therapy, or determining if a subject is having, is suspected of having, or having an increased risk for developing a disorder or a disease selected from Alzheimer's disease, Parkinson's disease, Parkinson's disease with dementia, Huntington's disease, Amyotrophic lateral sclerosis, Prion disease, diabetic cardiomyopathy, non-alcoholic fatty liver disease, a coronavirus disease, a disorder related to thermogenesis, a disorder related to oxidative phosphorylation, a disorder related to proteasome dysfunction, a disorder related to ribosome dysfunction, a disorder related to retrograde endocannabinoid signaling, or a disorder related to cardiac muscle contraction. In one aspect, the disease or disorder is Parkinson's disease, Parkinson's disease with dementia, or Prion disease.

A composition comprising a Sigma-1 receptor agonist as used in the kit may comprise ANAVEX2-73 (A2-73), A2-73 free base, ANAVEX 19-144, ANAVEX1-41, AV1066, ANAVEX3-71, PRE-084, L-687,384, SA-4503, Dimethyltryptamine, (+)-pentazocine, or any of their crystal forms, enantiomers and pharmaceutically acceptable salts thereof. For example, the composition is a A2-73 oral composition comprising A2-73 free base, A2-73 amorphous form, A2-73 Crystal Form I, A2-73 Crystal Form II, A2-73 Crystal Form III, (−) A2-73 enantiomer, (+) A2-73 enantiomer, or any combination thereof.

Another aspect of the present disclosure encompasses a method of treating a disorder or a disease in a subject by administering a therapeutically effective amount of a Sigma-1 receptor agonist. The disorder or disease comprises Alzheimer's disease, Parkinson's disease, Parkinson's disease with dementia, Huntington's disease, Amyotrophic lateral sclerosis, Prion disease, diabetic cardiomyopathy, non-alcoholic fatty liver disease, a coronavirus disease, a disorder related to thermogenesis, a disorder related to oxidative phosphorylation, a disorder related to proteasome dysfunction, a disorder related to ribosome dysfunction, a disorder related to retrograde endocannabinoid signaling, a disorder related to cardiac muscle contraction, or any combination thereof. The Sigma-1 receptor agonist may comprise ANAVEX2-73 (A2-73), A2-73 free base, ANAVEX 19-144, ANAVEX1-41, AV1066, ANAVEX3-71, PRE-084, Donepezil, Fluvoxamine, Amitriptyline, L-687,384, SA-4503, Dextromethorphan, Dimethyltryptamine, (+)-pentazocine, or any of their crystal forms, enantiomers and pharmaceutically acceptable salts thereof. For example, the Sigma-1 receptor agonist comprises A2-73 free base, A2-73 amorphous form, A2-73 Crystal Form I, A2-73 Crystal Form II, A2-73 Crystal Form III, (−) A2-73 enantiomer, (+) A2-73 enantiomer, or any combination thereof. And the therapeutically effective amount of A2-73 comprises A2-73 in an amount of 0.5-100 mg per day, such 10-50 mg per day. In one aspect, the treatment may be directed to treating Parkinson's Disease, and comprises administering an effective amount of A2-73 in about 10 mg to 50 mg per day. A2-73 may be in a form of free base, A2-73 amorphous form, A2-73 Crystal Form I, A2-73 Crystal Form II, A2-73 Crystal Form III, (−) A2-73 enantiomer, (+) A2-73 enantiomer, or any combination thereof. In another aspect, the treatment may be directed to treating Prion Disease and comprises administering A2-73 in an amount of 0.5-100 mg per day. A2-73 may be in a form of free base, A2-73 amorphous form, A2-73 Crystal Form I, A2-73 Crystal Form II, A2-73 Crystal Form III, (−) A2-73 enantiomer, (+) A2-73 enantiomer, or any combination thereof. The subject being treated may be a human subject or a non-human mammal.

Another aspect of the present disclosure encompasses a method of regulating expression of gene MS4A4A in a subject in need thereof, the method comprising administering the subject with a Sigma receptor agonist. In some aspect, the subject is having or is suspect of having a microglia dysfunction. Such microglia dysfunction may manifest a neurodegenerative disorder, such neurodegenerative disorder comprises Alzheimer's disease, Parkinson's disease, Parkinson's disease with dementia, Huntington's disease, Amyotrophic lateral sclerosis, Prion disease, and any combination thereof.

In yet another aspect, the present disclosure encompasses a method of regulating expression of gene MS4A4A by administering a Sigma receptor agonist, such as ANAVEX2-73 (A2-73), A2-73 free base, ANAVEX 19-144, ANAVEX1-41, AV1066, ANAVEX3-71, PRE-084, Donepezil, Fluvoxamine, Amitriptyline, L-687,384, SA-4503, Dextromethorphan, Dimethyltryptamine, (+)-pentazocine, and any crystal forms, enantiomers and pharmaceutically acceptable salts thereof. In another aspect, the Sigma receptor agonist comprises A2-73 free base, A2-73 amorphous form, A2-73 Crystal Form I, A2-73 Crystal Form II, A2-73 Crystal Form III, (−) A2-73 enantiomer, or (+) A2-73 enantiomer. In yet another aspect, the Sigma receptor agonist is administered daily. In another aspect, the Sigma receptor agonist comprises A2-73 free base or A2-73 amorphous form, and wherein the A2-73 is administered daily in an amount of about 10-100 mg for a period of 6 to 14 weeks. In another aspect, the Sigma receptor agonist comprises A2-73 in a crystal form or an optical pure form, and wherein the A2-73 is administered once daily in an amount of about 40-60 mg for a period of up to 11 weeks. In another aspect, the Sigma receptor agonist comprises A2-73 in a crystal form or an optical pure form, and wherein the A2-73 is administered once daily for a period of 11 weeks in an escalating amount starting at about 10 mg and ending at about 60 mg.

In another aspect, the present disclosure encompasses a method of evaluating effectiveness of a neurodegenerative therapy in a subject in need thereof. The method comprising the steps of (a) obtaining or having obtained a first biological sample of the subject before onset of the neurodegenerative therapy; (b) providing the neurodegenerative therapy to the subject for a period of therapy time; (c) obtaining or having obtained a second biological sample of the subject at the end of the therapy time; (d) determining MS4A4A gene expression level in the first biological sample and that in the second biological sample; and (e) identifying the neurodegenerative therapy as (i) effective for the subject, if the MS4A4A gene expression level in the first biological sample is lower than that in the second biological sample; or (ii) ineffective for the subject, if the MS4A4A gene expression level in the first biological sample is higher than that in the second biological sample. In another aspect, if the neurodegenerative therapy is identified as ineffective in (e), the method further comprises one or more of the following: i. switching to another neurodegenerative therapy; ii. supplementing with another neurodegenerative therapy; iii. adjusting dose if the neurodegenerative therapy involves drug therapy; and iv. switching to a combined neurodegenerative therapy.

In another aspect, the first biological sample or the second biological sample is each independently comprises a blood serum sample, a blood plasma sample, or a sample suitable for transcriptomic analysis. In yet another aspect, the neurodegenerative therapy is for treating a disease selected from Alzheimer's disease, Parkinson's disease, Parkinson's disease with dementia, Huntington's disease, Amyotrophic lateral sclerosis, Prion disease, and any combination thereof. In yet another aspect, the neurodegenerative therapy comprises a drug therapy selected from ANAVEX2-73 (A2-73), ANAVEX 19-144, ANAVEX1-41, AV1066, ANAVEX3-71, PRE-084, Donepezil, Fluvoxamine, Amitriptyline, L-687,384, SA-4503, Dextromethorphan, Dimethyltryptamine, (+)-pentazocine, and any of their crystal forms, enantiomers and pharmaceutically acceptable salts. In yet another aspect, the neurodegenerative therapy is selected from a drug therapy comprising A2-73 free base, A2-73 amorphous form, A2-73 Crystal Form I, A2-73 Crystal Form II, A2-73 Crystal Form III, (−) A2-73 enantiomer, and (+) A2-73 enantiomer. In yet another aspect, the period of therapy time is up to 14 weeks. In another aspect, the period of therapy time is about 4 weeks, about 5 weeks, about 6 weeks, about 7 weeks, about 8 weeks, about 9 weeks, about 10 weeks, about 11 weeks, about 12 weeks, about 13 weeks, or about 14 weeks. In another aspect, the neurodegenerative therapy is administered daily. In another aspect, the neurodegenerative therapy comprises a A2-73 drug therapy, and wherein the A2-73 drug therapy comprises daily administering A2-73 or A2-73 free base in an amount of about 10-100 mg, and for a period of 6 to 14 weeks. In another aspect, the neurodegenerative therapy comprises a A2-73 drug therapy, and wherein the A2-73 drug therapy comprises administering a crystal form or an optical pure form of A2-73 once daily. In another aspect, the neurodegenerative therapy comprises a A2-73 drug therapy, and wherein the A2-73 drug therapy comprises administering a crystal form of A2-73 once daily in an escalating amount starting at about 10 mg and ending about 50 mg, and for a period up to 14 weeks. In another aspect, the neurodegenerative therapy comprises a A2-73 drug therapy, and wherein the A2-73 drug therapy comprises administering an optical form of A2-73 once daily in an escalating amount starting at about 10 mg and ending about 60 mg, and for a period up to 14 weeks.

Another aspect of the present disclosure encompasses a method of identifying a subject responsive to a Sigma-1 receptor agonist therapy, the method comprising the steps of (a) obtaining or having obtained a biological sample of the subject; (b) determining a genetic profile from the biological sample; (c) identifying gene MS4A4A in the genetic profile; (d) detecting coding region of the gene MS4A4A; (e) determining if the coding region possess rs6591561 Wild Type; and (f) identifying the subject being responsive to the Sigma-1 receptor agonist therapy to determine if the subject possesses rs6591561 Wild Type. In one aspect, the biological sample comprises a blood serum sample, a blood plasma sample, or a sample suitable for gene analysis. In another aspect, the Sigma-1 receptor agonist therapy is a drug therapy selected from ANAVEX2-73 (A2-73), A2-73 free base, ANAVEX 19-144, ANAVEX1-41, AV1066, ANAVEX3-71, PRE-084, L-687,384, SA-4503, Dimethyltryptamine, (+)-pentazocine, and any of their crystal forms, enantiomers and pharmaceutically acceptable salts. In some aspect, the Sigma-1 receptor agonist therapy is a A2-73 drug therapy comprising administration of A2-73 free base, A2-73 amorphous form, A2-73 Crystal Form I, A2-73 Crystal Form II, A2-73 Crystal Form III, (−) A2-73 enantiomer, (+) A2-73 enantiomer, or any combination thereof.

Another aspect of the present disclosure encompasses the foregoing treatment of a human subject having or being suspected of having a disorder selected from Alzheimer's disease, Parkinson's disease, Parkinson's disease with dementia, Huntington's disease, Amyotrophic lateral sclerosis, Prion disease, diabetic cardiomyopathy, non-alcoholic fatty liver disease, a coronavirus disease, a disorder related to thermogenesis, a disorder related to oxidative phosphorylation, a disorder related to proteasome dysfunction, a disorder related to ribosome dysfunction, a disorder related to retrograde endocannabinoid signaling, a disorder related to cardiac muscle contraction, and any combination thereof. In some aspect, the Sigma-1 receptor agonist therapy is provided daily. In another aspect, the Sigma-1 receptor agonist therapy comprises a A2-73 drug therapy, and the A2-73 drug therapy comprises daily administering of A2-73 or A2-73 free base in an amount of about 40-60 mg, and for a period of 6 to 14 weeks. In some aspect, the Sigma-1 receptor agonist therapy is a A2-73 drug therapy, and wherein the A2-73 drug therapy comprises administering a crystal form of A2-73 or A2-73 freebase once daily in an amount of about 50 mg, and for a period of up to 11 weeks. In some aspect, the Sigma-1 receptor agonist therapy comprises a A2-73 drug therapy, and wherein the A2-73 drug therapy comprises administering is a crystal form of A2-73 or A2-73 free base once daily for a period of 11 weeks in an escalating amount starting at about 10 mg and ending at about 50 mg.

Another aspect of the present disclosure encompasses a method of determining if a subject is having or is suspected of having a microglia dysfunction, the method comprising the steps of (a) obtaining or having obtained a first biological sample of the subject; (b) administering to the subject a composition of A2-73 for a period of time; (c) obtaining or having obtained a second biological sample of the subject at the end of the period of time; (d) determining a gene expression profile for the first biological sample and for the second biological sample; and (e) comparing gene expression level of MS4A4A in the first biological sample with that in the second biological sample. It is to confirm the subject having the microglia dysfunction, if the gene expression level of MS4A4A is increased. In some aspect, first biological sample and the second biological sample comprise independently a blood serum sample, a blood plasma sample, or a sample suitable for gene analysis. In some aspect, the microglia dysfunction manifests a disease selected from the group consisting of Alzheimer's disease, Parkinson's disease, Parkinson's disease with dementia, Huntington's disease, Amyotrophic lateral sclerosis, Prion disease, diabetic cardiomyopathy, non-alcoholic fatty liver disease, a coronavirus disease, a disorder related to thermogenesis, a disorder related to oxidative phosphorylation, a disorder related to proteasome dysfunction, a disorder related to ribosome dysfunction, a disorder related to retrograde endocannabinoid signaling, and a disorder related to cardiac muscle contraction. In yet another aspect, the composition of A2-73 comprises A2-73 free base, A2-73 amorphous form, A2-73 Crystal Form I, A2-73 Crystal Form II, A2-73 Crystal Form III, (−) A2-73 enantiomer, (+) A2-73 enantiomer, or any combination thereof. In yet another aspect, the composition of A2-73 comprises an oral composition. In another aspect, the period of time is up to 14 weeks. In yet another aspect, the period of time is about 4 weeks, about 5 weeks, about 6 weeks, about 7 weeks, about 8 weeks, about 9 weeks, about 10 weeks, about 11 weeks, about 12 weeks, about 13 weeks, or about 14 weeks. In yet another aspect, the A2-73 composition is administered daily. In another aspect, the A2-73 composition is administered daily in an amount of about 40-60 mg, and for a period of 6 to 14 weeks. In another aspect, the A2-73 composition comprises a crystal form of A2-73 or A2-73 free base, and is administered once daily in an amount of about 50 mg, and for a period of up to 11 weeks. In another aspect, the A2-73 compositions comprises a crystal form of A2-73 or A2-73 free bases, and is administered once daily in an escalating amount starting at about 10 mg and ending about 50 mg, and for a period of up to 11 weeks.

Another aspect of the present disclosure encompasses a method of assessing a risk of a subject for developing a disease related to microglia dysfunction, the method comprising the steps of (a) obtaining or having obtained a biological sample from the subject; (b) identifying MS4A4A gene expression in the biological sample; (c) detecting coding region of MS4A4A; (d) determining if the coding region possess rs6591561 Wild Type or rs1582763 Wild Type; and (e) concluding the subject (i) possessing rs6591561 Wild Type has a higher risk of developing the disease related to microglia dysfunction than those without the rs6591561 Wild Type; or (ii) possessing rs1582763 Wild Type has a lower risk of developing the disease related to microglia dysfunction than those with the rs6591561 Wild Type. In some aspect, the biological sample comprises a blood serum sample, a blood plasma sample, or a sample suitable for gene analysis. In another aspect, the disease related to microglia dysfunction comprises Alzheimer disease, Parkinson's disease, or Parkinson's disease with dementia.

Another aspect of the present disclosure encompasses A2-73 for use as a medicament in the treatment of a microglia dysfunction wherein the microglia dysfunction is caused by the presence of MS4A4A variant rs6591561.

Another aspect of the present disclosure encompasses a method of treating a microglia dysfunction in a subject in need thereof, comprising administering to the subject a therapeutically effective amount of A2-73, and the therapeutically effective amount is an amount sufficient to transition microglia to an anti-inflammatory (Mic 3) state with efficient lipid metabolism. In some aspect, the A2-73 is A2-73 free base, crystal Form I of A2-73, crystal Form II of A2-73, crystal Form III of A2-73, (−) enantiomer of A2-73, (+) entantiomer of A2-73, or any combination thereof. In yet another aspect, the A2-73 is in an amount of 0.5-100 mg per day. And in some aspect, the microglia dysfunction is Parkinson's Disease and the A2-73 is in an oral amount of 1-100 mg per day. In yet another aspect, the microglia dysfunction is Parkinson's Disease with dementia and the A2-73 is in an oral amount of 10-50 mg per day.

Another aspect of the present disclosure encompasses a method of identifying a disease impactful biomarker through artificial intelligence modeling, the method comprises the steps of (a) designing or having designed an interventional clinical trial to study therapeutic effect of a drug candidate to the disease, wherein the clinical trial specifies a protocol of the drug candidate to be administered, a biomedical information to be collected and time intervals for such administration and collection; (b) enrolling or having enrolled a population of patients having the disease into the interventional clinical trial; (c) gathering or having gathered a set of drug administration information and biomedical information based on the designed clinical trial and at the time intervals specified in the designed clinical trial; (d) generating relationships from the gathered drug administration information and biomedical information; (e) characterizing the generated relationships; (f) identified from the characterized relationships a biomarker with confidence level over 0.5, Lift over 1, and Fisher's p value less than <0.08; and (g) designating the biomarker as a disease-impactful biomarker. In one aspect, the gathered drug administration information and biomedical information comprises at least patients' clinical diagnosis, co-morbidity, co-medication, age, weight, height, gender, dose received, dosing time points, endpoint assessment, and omics. In one aspect, the drug candidate is A2-73. In yet another aspect, the drug candidate comprises A2-73 in a crystal form, in an optical pure form, or as A2-73 freebase. In yet another aspect, the drug candidate comprises A2-73 Crystal Form I, A2-73 Crystal Form II, A2-73 Crystal Form III, (−) A2-73 enantiomer, (+) A2-73 enantiomer, or a combination thereof. In one aspect, the disease is Alzheimer disease, Parkinson's disease, or Parkinson's disease with dementia. In one aspect, the biomarker identified comprises MS4A4A gene. In yet another aspect, the biomarker identified comprises MS4A4A gene possessing single nucleotide polymorphism rs6591561 or rs1582763. In yet another aspect, the biomarker identified comprises MS4A4A rs6591561.

### REFERENCE TO COLOR FIGURES

The application file contains at least one drawing or photograph executed in color. Copies of this patent application publication with color drawings or photographs will be provided by the Office upon request and payment of the necessary fee.

### BRIEF DESCRIPTION OF THE FIGURES

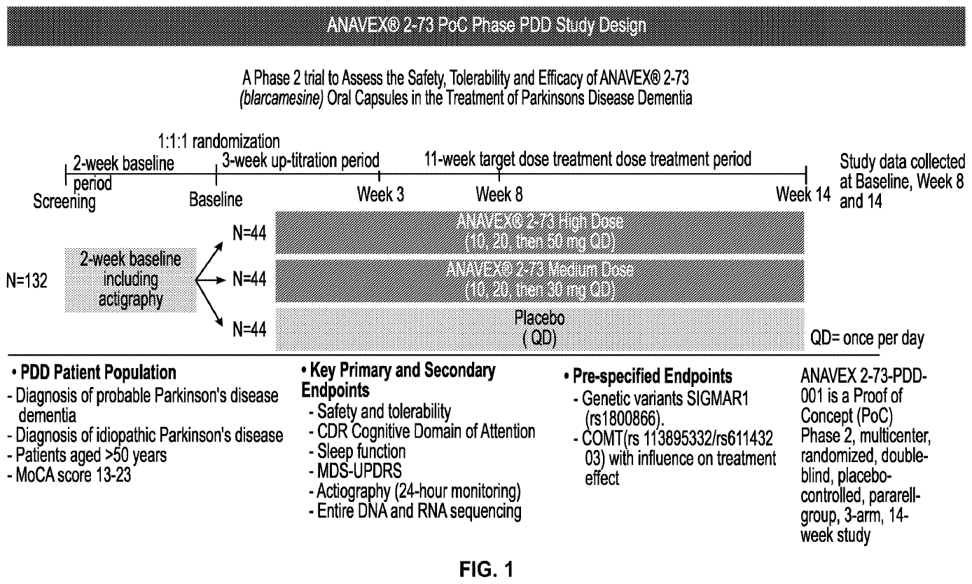

FIG. 1 is an illustrative overview of Phase 2 Proof of Concept (PoC) study design of ANAVEX® 2-73 for treating Parkinson's Disease with Dementia (PDD), including patient selection, endpoints information and analytical methods.

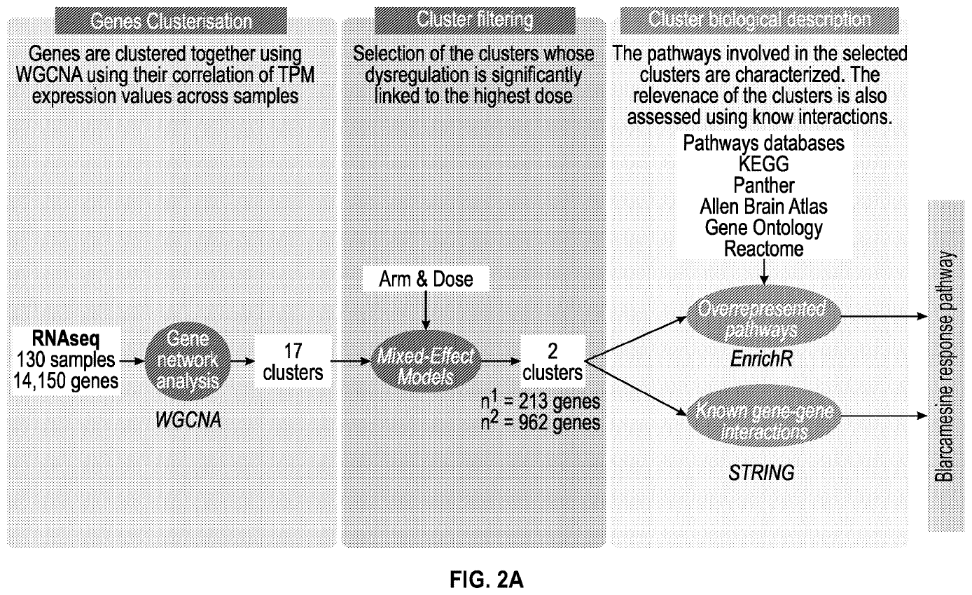

FIG. 2A-2B are illustrative overview on multi-disciplinary method to identify genes response pathways of ANAVEX® 2-73 treatment. In genes clusterization, genes were clustered together using WGCNA1 (Weighted correlation network analysis. Correlation networks are being used in biology to analyze large, high-dimensional data sets), starting from their correlation of TPM2 expression (Transcript per million. TPM value represents a relative expression level that should be comparable between samples) values across samples: WGCNA created 17 clusters (size: 13 to 11,190 genes). Then, the cluster's eigengene3 (one of a set of a genes matrix that tabulates the mRNA or gene expression of the genes across the sample) was used as summary of expression level of the cluster. In cluster filtering, the eigengene was used for each cluster as target variable of a linear mixed effect model with 3 covariates: Dose, patient, and timepoint. Clusters without significant (Dunnett's test) contrasts between treated and placebo across all timepoints were filtered out. The ratio between cluster size and number of pathways was calculated and the top 2 clusters were eventually retained. In clusters biological description, two approaches were studied. FIG. 2A showed characterization and analysis were applied to all the genes of the cluster (whole cluster), and to the genes of the clusters that were known to have biological interactions (reduced cluster). In the whole cluster analysis, overrepresented pathways were identified using EnrichR package with the parameters of Fisher's adjusted p-value p<0.05 (Benjamini-Hochberg adjustment for multiple testing) and minimum pathway size 20. In the reduced cluster analysis, genes that have no interaction with any other genes of the cluster were discarded, resulting in a reduced cluster. Pathway analysis was performed on these reduced clusters. False Discovery Rate with adjustment for multiple testing (Benjamini-Hochberg procedure) was used to characterize pathways' over-representation. Known interactions between the genes of the cluster were assessed using STRING database with the parameters of Edge score >0.150, and no text mining edges. Known interactions were obtained from public databases, such as KEGG, Panther, Allen, Brain Atlas, Gene Ontology, and Reactome. Alternatively, FIG. 2B showed each cluster's functional interactions between the genes of the cluster were assessed using the STRING database (Edge score >0.150, no text mining edges). Genes that have no interaction with any other genes of the cluster were discarded, which led to a reduced cluster. Pathway enrichment analysis was performed on these reduced clusters. False Discovery Rate (FDR, adjustment for multiple testing with the Benjamini-Hochberg procedure) with adjustment for multiple testing (Benjamini-Hochberg procedure) was used to characterize pathway overrepresentation.

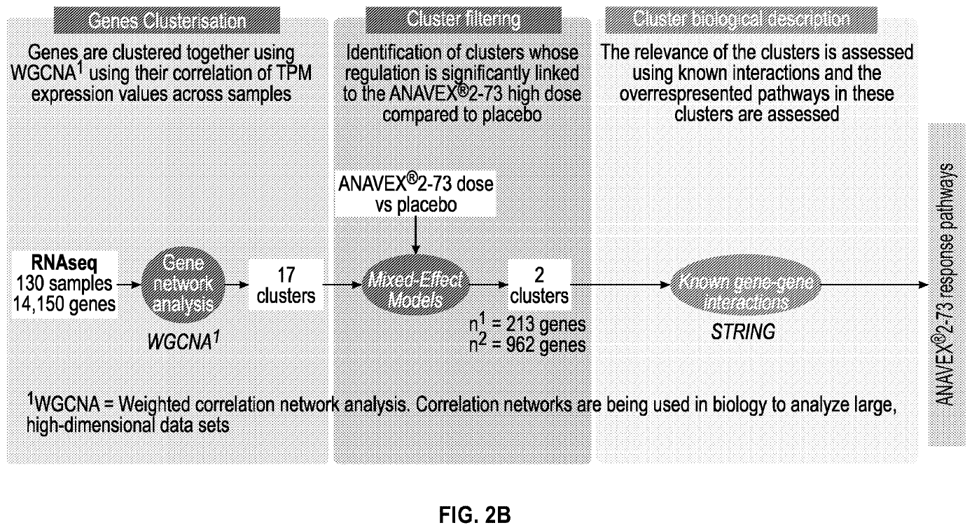

FIG. 3 is a log P value plot of estimated marginal means of the mixed model, wherein the P value is from Dunnett's test and linear mixed-effect model has 3 covariates, Actual Dose, Patient, and timepoint.

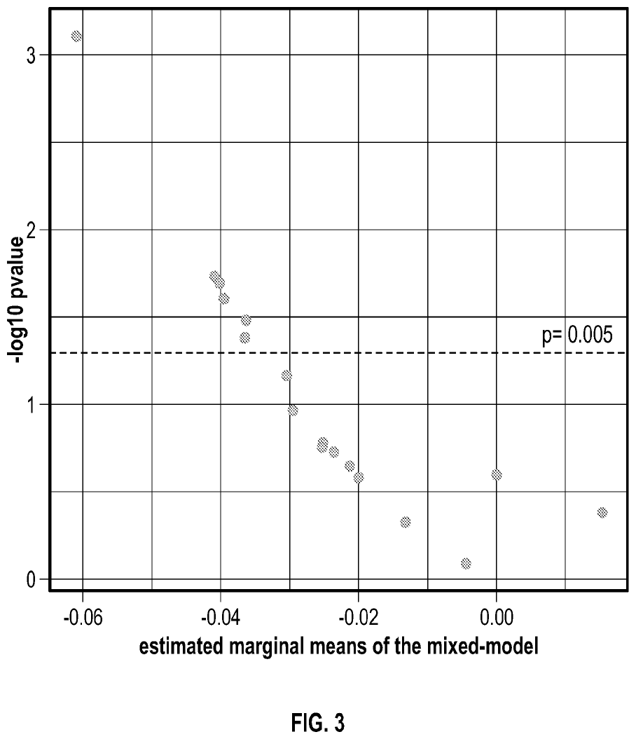

FIG. 4A-4B are interaction plots of genes in Cluster 1 with 213 genes, wherein p-values were calculated by Fisher's test. FIG. 4A shows genes involved in the biological pathways of Parkinson's Disease, Prion Disease and Huntington's disease. FDR stands for False Discovery Rate, adjustment for multiple testing with the Benjamini-Hochberg procedure. Only edges with score >0.4 were shown. FIG. 4B plots genes in KEGG 2022 pathways, and lists genes involved in the biological pathways of Parkinson's Disease, Alzheimer's Disease and other neurodegenerative diseases. It showed among 213 genes, 79% of the genes (169) have biological interactions.

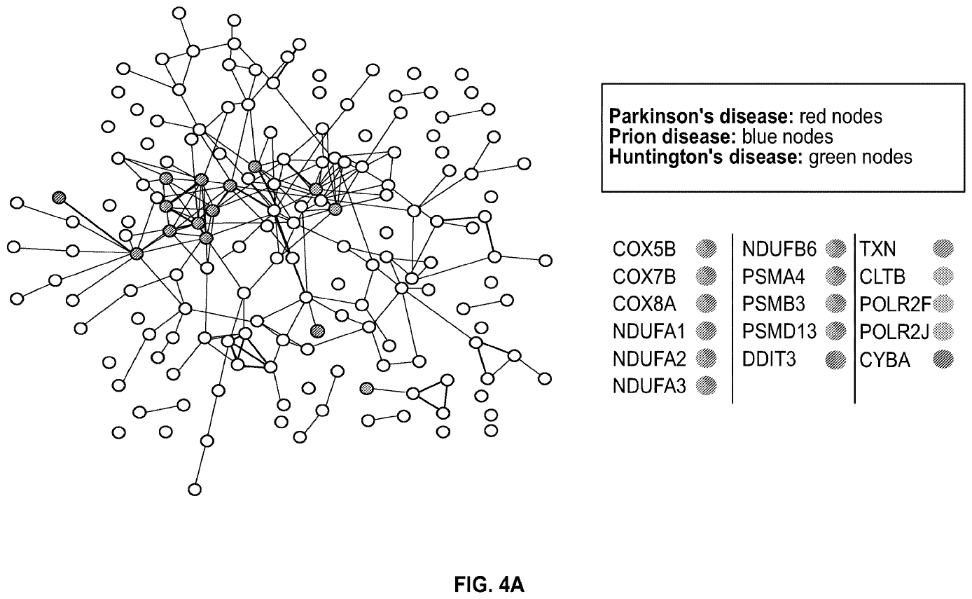

FIG. 5A-5B are interaction plots of genes in Cluster 2 with 962 genes and involved in the biological pathway of degenerative diseases. FDR stands for False Discovery Rate, adjustment for multiple testing with the Benjamini-Hochberg procedure. FIG. 5A shows and lists genes with interaction score >0.4. FIG. 5B shows among the 962 genes, 70% of the genes (680) have biological interactions correlating to Parkinson's Disease, Alzheimer's Disease and other neurodegenerative diseases.

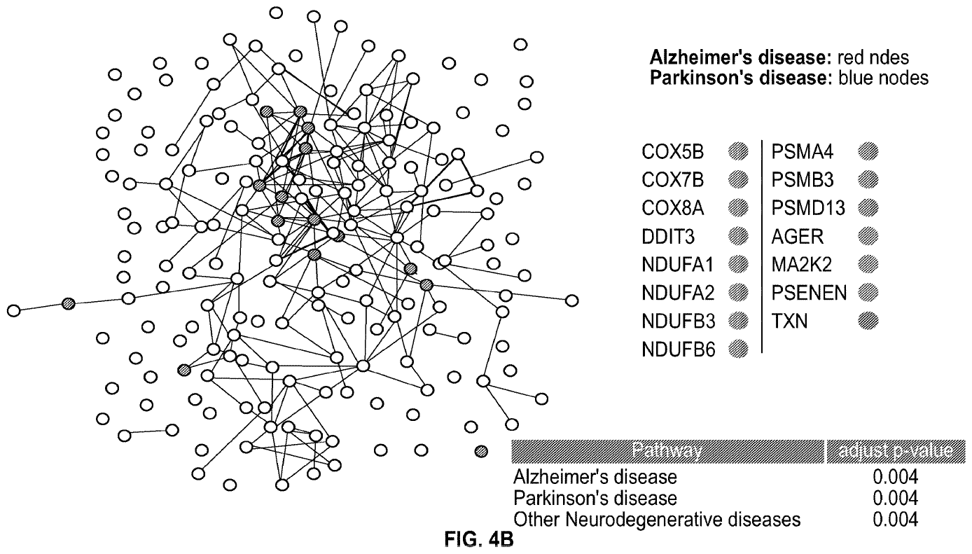

FIG. 6 depicts the features and progress of Parkinson's Disease (PD).

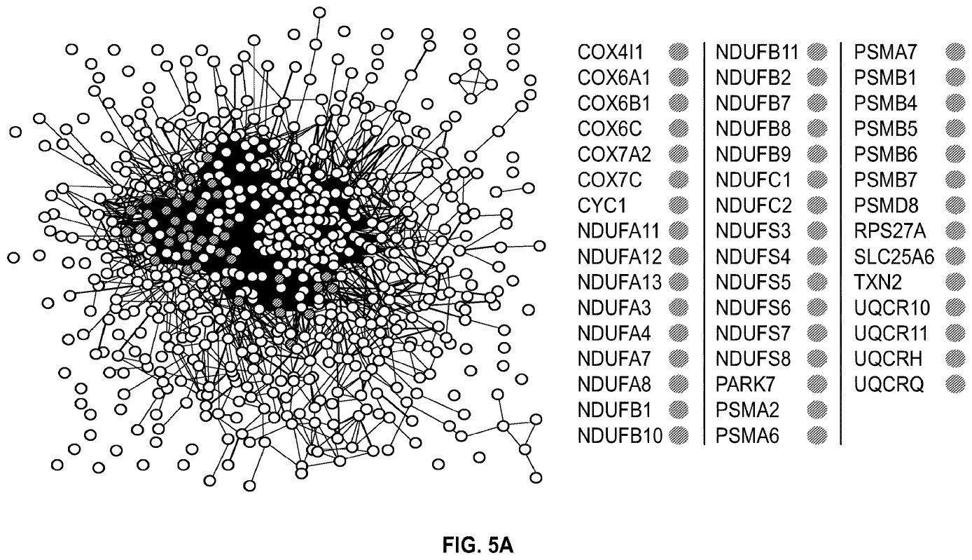

FIG. 7 depicts PD and Parkinson's Disease with Dementia (PDD) related endpoints incorporated in PDD-001 Phase 2 study, illustrating the potential effects of ANAVEX® 2-73 on both PD and PDD.

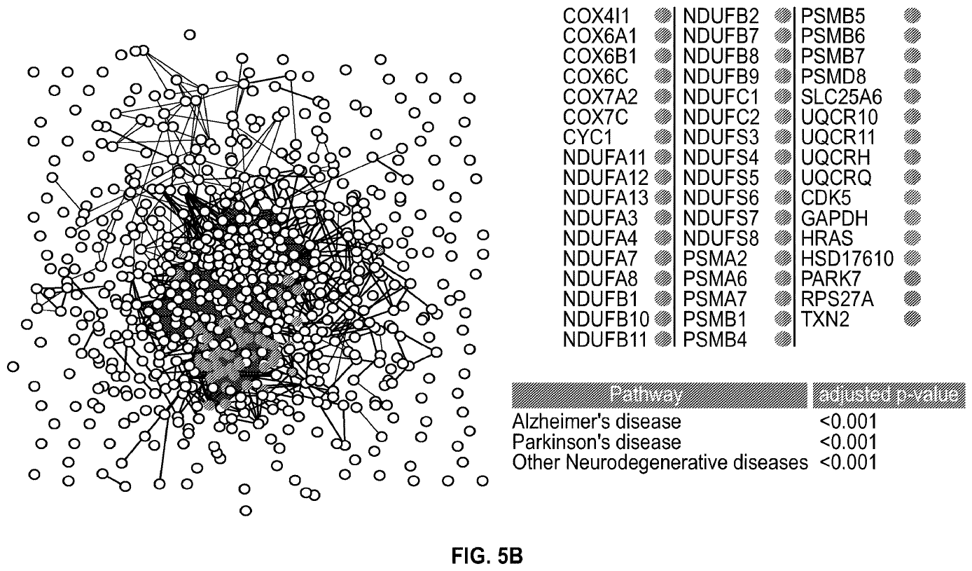

FIG. 8 depicts the Artificial Intelligence (AI) analysis using Computerized Models to identify new biomarkers.

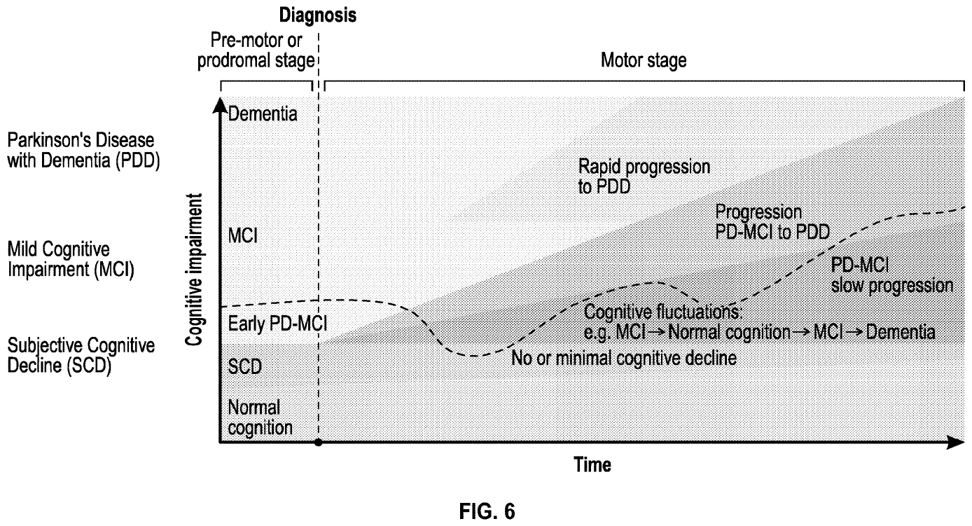

FIG. 9 depicts microglia transitions to either a pro-inflammatory (Mic 1) or anti-inflammatory (Mic 3) state. Left portion shows interferon-based response with efficient lipid metabolism. Right portion shows a destructive inflammatory response that accumulates lipids.

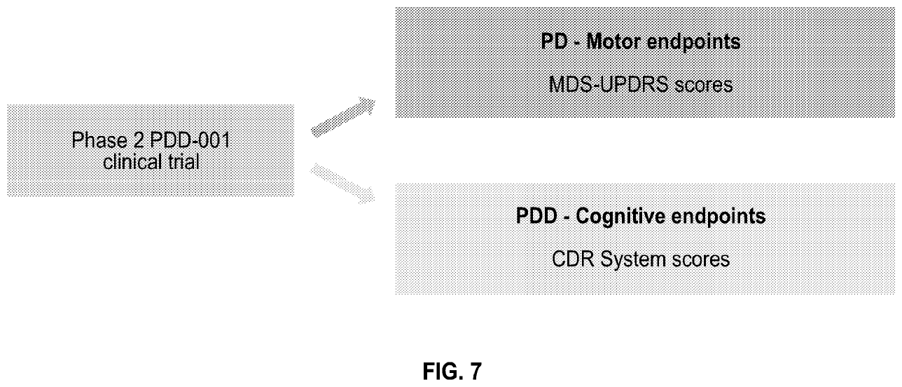

FIG. 10 depicts the All-Corners Studies for biomarker selection of patient populations in targeted medicine.

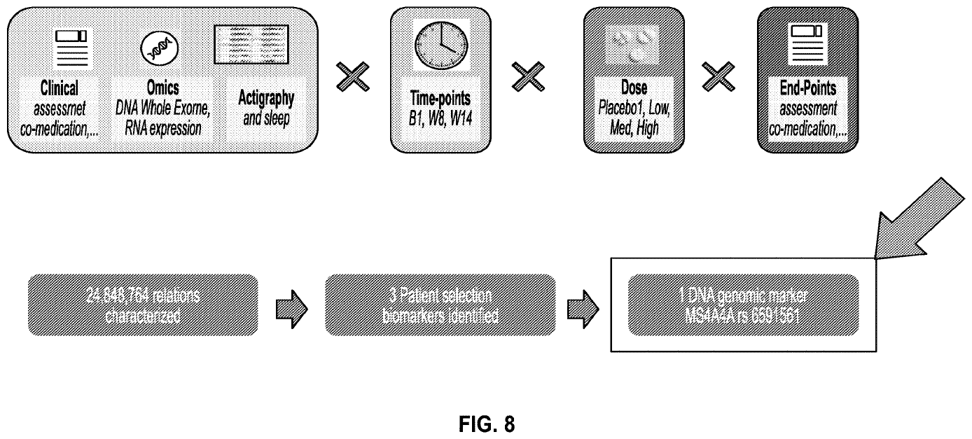

FIG. 11 depicts Biomarker Noise Model, showing biomarker selection of patient populations in targeted medicine for reproducible therapeutic effect.

### DETAILED DESCRIPTION

The present disclosure is based in part on the discovery that when a subject is for a period of time administered a therapeutic agent such as a Sigma-1 receptor agonist, a set of selected genes are differentially expressed. Further, these genes are involved in distinct biological pathways implicated in certain diseases and disorders. The discovered correlation between disease treatment and changes in genetic profiles pre- and post-treatment, such as differentially expressed genes and/or overrepresented gene clusters, can be used to further guide choices concerning the treatment. For instance, the correlation may be used to evaluate therapeutic effectiveness of another therapeutic agent, or to select a therapeutic agent for a subject based on changes induced in genetic profiling, or to assess a subject's disease state or risk for developing a disease using a therapeutic agent as a probe. The present disclosure is focused on Sigma receptor agonist therapy, and it is related diseases and disorders. But the same principle and methods can be similarly applied to other classes of therapeutic agents and diseases.

Sigma-1 receptors are shown to be involved in higher-ordered brain functions including memory and cognition. Thus, a Sigma-1 receptor agonist therapy is often prescribed to patients having declined memory or cognition functions, such as those with neurodegenerative disorders. It is discovered that after a subject is being treated with a Sigma-1 receptor agonist, his or her gene expression profiles are altered: a set of selected genes are differentially expressed, and their relevant gene clusters are overrepresented. The altered genetic profile can be used as a benchmark to evaluate the therapeutic effect of another therapeutic agent. It can also be used to select a Sigma-1 receptor agonist for a subject. Further, it can also be used to determine if a subject is responsive to a Sigma-1 receptor agonist therapy. Finally, the Sigma-1 receptor agonist can be used a probe to see if a subject is having, or is suspected of having, or at an increased risk of having, a disease linked to the altered gene expression profiles.

1. Gene Profile Changes Associated with Sigma-1 Receptor Agonist Therapy

The inventors have discovered that a significant number of genes are differentially expressed after a period of Sigma-1 receptor agonist therapy. These genes are split into two clusters. The first gene cluster comprises MS4A4A, COX5B, COX7B, COX8A, NDUFB6, TXN, PSMA4, CLTB, PSMB3, POLR2F, NDUFA1, PSMD13, POLR2, NDUFA2, DDIT3, CYBA, AGER, MAPA2K2, PSENEN, and NDUFB3. The second gene cluster comprises COX4I1, NDUFB11, PSMA7, COX6A1, NDUFB2, PSMB1, COX6B1, NDUFB7, PSMB4, COX6C, NDUFB8, PSMB5, COX7A2, NDUFB9, PSMB6, COX7C, NDUFC1, PSMB7, CYC1, NDUFC2, PSMD8, NDUFA11, NDUFS3, RPS27A, NDUFA12, NDUFS4, SLC25A6, NDUFA13, NDUFS5, TXN2, NDUFA3, NDUFS6, UQCR10, NDUFA4, NDUFS7, UQCR11, NDUFA7, NDUFS8, UQCRH, NDUFA8, PARK7, UQCRQ, NDUFB1, PSMA2, NDUFB10, CDK5, GAPDH, HRAS, HSD17B10, or PSMA6. Both clusters present some overrepresented pathways, which are related to various biological processes, dysfunctions and diseases, such as neurodegenerative diseases and metabolic disorders. For example, the first gene cluster is found to have correlation with nine (9) diseases or dysfunctions, and five (5) of them are neurodegenerative diseases. The nine (9) diseases or dysfunctions are Alzheimer's disease, Parkinson's disease, Parkinson's disease with dementia, Huntington's disease, Amyotrophic lateral sclerosis, Prion disease, diabetic cardiomyopathy, non-alcoholic fatty liver disease, oxidative phosphorylation dysfunction, and proteasome dysfunction. The second gene cluster is found to have correlation with fourteen (14) biological functions, diseases or dysfunctions, and five (5) of them are neurodegenerative diseases, such as Alzheimer's disease, Parkinson's disease, Parkinson's disease with dementia, Huntington's disease, Amyotrophic lateral sclerosis, Prion disease, coronavirus disease, diabetic cardiomyopathy, non-alcoholic fatty liver disease, oxidative phosphorylation dysfunction, ribosome dysfunction, proteasome function, and thermogenesis function. It is found MS4A4A gene regulates lipid metabolism and immune response in a unique microglia state. For example, in disease conditions, homeostatic microglia can transition to either a pro-inflammatory (Mic 1) or anti-inflammatory (Mic 3) state. Mic 3 cells are themselves influenced by protective and risk single nucleotide polymorphisms (SNPs) in the MS4A4A locus, which push these cells into either a helpful, interferon-based response with efficient lipid metabolism, or a destructive inflammatory response that accumulates lipids.

Neurodegenerative diseases refer to hereditary and sporadic conditions which are characterized by progressive nervous system dysfunction. These disorders are often associated with atrophy of the central or peripheral nervous system structures and may have symptoms in the form of cognition impairment, memory loss, and movement disorders. Alzheimer's disease is a degenerative disease of the brain characterized by the insidious onset of dementia. Early symptoms include impairment of memory, judgment, attention span, and problem-solving skills. In late stage, severe apraxias and a global loss of cognitive abilities may occur. The disease is marked pathologically by severe cortical atrophy and the triad of senile plaques, neurofibrillary tangles, and neuropil threads.

Parkinson's Disease (PD) is a highly heterogeneous multisystem disorder, it is the second most common neurodegenerative disorder where also cognitive impairment is common. It is considered the second largest CNS disorder with over 10,000 patients worldwide. It is fairly common in older adults, estimated to affect nearly 2 percent of those older than age 65. It is estimated that PD prevalence in US is over 1,000,000 patients. PD is a progressive, degenerative neurologic disease characterized by a tremor that is maximal at rest, retropulsion (i.e. a tendency to fall backwards), rigidity, stooped posture, slowness of voluntary movements, and a masklike facial expression. Pathologic features include loss of melanin containing neurons in the substantia nigra and other pigmented nuclei of the brainstem. The brain changes caused by Parkinson's disease begin in a region that plays a key role in movement. But etiology of cognitive impairment in PD has not yet been fully elucidated. As Parkinson's brain changes gradually spread, they often begin to affect mental functions, including memory and the ability to pay attention, make sound judgments and plan the steps needed to complete a task. Lewy bodies are present in the substantia nigra and locus coeruleus but may also be found in a related condition characterized by dementia in combination with varying degrees of parkinsonism. Symptoms of Parkinson's disease may include, but not limited to, tremors and shakiness, muscle stiffness, a shuffling step, stooped posture, difficulty initiating movement, lack of facial expression, and dementia. Parkinson's Disease with Dementia (PDD) is a decline in thinking and reasoning skills that develops in some people living with Parkinson's disease. Parkinson patients may have movement problems before cognitive symptoms in dementia associated with Parkinson's disease. In dementia with Lewy bodies, cognitive symptoms occur before, or at the same time as, problems with movement. Secondary Parkinson's disease refers to conditions which feature clinical manifestations resembling primary Parkinson's disease that are caused by a known or suspected condition, such as parkinsonism caused by vascular injury, drugs, trauma, toxin exposure, neoplasms, infections and degenerative or hereditary conditions. Clinical features may include bradykinesia, rigidity, parkinsonian gait, and masked facies. In general, tremor is less prominent in secondary parkinsonism than in the primary form. As used herein, Parkinson's disease expressly incorporates the pathological features, the clinical manifestations, the symptoms and any secondary Parkinson's disease. Specifically, Parkinson disease used herein expressly incorporates Parkinson's Disease with Dementia (PDD).

Huntington's disease is a familial disorder inherited as an autosomal dominant trait and characterized by the onset of progressive chorea and dementia in the fourth or fifth decade of life. Common initial manifestations include paranoia, poor impulse control, depression, hallucinations, and delusions. Late symptoms include intellectual impairment, loss of fine motor control, athetosis, and diffuse chorea involving axial and limb musculature develops, leading to a vegetative state within 10-15 years of disease onset. Its juvenile variant has a more fulminant course including seizures, ataxia, dementia, and chorea. Amyotrophic lateral sclerosis is a degenerative disorder affecting upper motor neurons in the brain and lower motor neurons in the brain stem and spinal cord. Disease onset is usually after the age of 50 and the process is usually fatal within 3 to 6 years. Clinical manifestations include progressive weakness, atrophy, fasciculation, hyperreflexia, dysarthria, dysphagia, and eventual paralysis of respiratory function. Pathologic features include the replacement of motor neurons with fibrous astrocytes and atrophy of anterior spinal nerve roots and corticospinal tracts. Prion disease is a group of genetic, infectious, or sporadic degenerative human and animal nervous system disorders associated with abnormal prions. These diseases are characterized by conversion of the normal prion protein to an abnormal configuration via a post-translational process. In humans, these conditions generally feature dementia, ataxia, and a fatal outcome. Pathologic features include a spongiform encephalopathy without evidence of inflammation. Some older literature may occasionally refer to these as unconventional slow virus diseases.

Prion disease, also called “transmissible spongiform encephalopathies” (TSEs), refers to a family of rare progressive neurodegenerative disorders that affect both humans and animals. They are distinguished by long incubation periods, characteristic spongiform changes associated with neuronal loss, and a failure to induce inflammatory response. The causative agents of TSEs are believed to be prions. The term “prions” refers to abnormal, pathogenic agents that are transmissible and are able to induce abnormal folding of specific normal cellular proteins called prion proteins that are found most abundantly in the brain. The abnormal folding of the prion proteins leads to brain damage and the characteristic signs and symptoms of the disease. Prion diseases are usually rapidly progressive and always fatal. Human Prion disease comprises at least Creutzfeldt-Jakob Disease (CJD), Variant Creutzfeldt-Jakob Disease (vCJD), Gerstmann-Straussler-Scheinker Syndrome, Fatal Familial Insomnia and Kuru. Animal Prion disease comprises at least Bovine Spongiform Encephalopathy (BSE), Chronic Wasting Disease (CWD), Scrapie, Transmissible mink encephalopathy, Feline spongiform encephalopathy, and Ungulate spongiform encephalopathy.

Amyotrophic lateral sclerosis, or “ALS”, often also called Lou Gehrig's disease (after the baseball player who was diagnosed with it). It is a progressive nervous system disease that affects nerve cells in the brain and spinal cord, causing loss of muscle control. ALS often begins with muscle twitching and weakness in a limb, or slurred speech. Its impact often starts in the hands, feet or limbs, and then spreads to other parts of your body. As the disease advances and nerve cells are destroyed, your muscles get weaker. This eventually affects chewing, swallowing, speaking and breathing. Symptoms include, but not limited to, difficulty walking or doing normal daily activities, tripping and falling, weakness in your legs, feet or ankles, hand weakness or clumsiness, slurred speech or trouble swallowing, muscle cramps and twitching in your arms, shoulders and tongue, inappropriate crying, laughing or yawning, and cognitive and behavioral changes. There is no cure for this fatal disease.

Diabetic cardiomyopathy (DCM) is defined by the existence of abnormal myocardial structure and performance in the absence of other cardiac risk factors, such as coronary artery disease, hypertension, and significant valvular disease, in individuals with diabetes mellitus. A change in the metabolic status, impaired calcium homeostasis and energy production, increased inflammation and oxidative stress, as well as an accumulation of advanced glycation end products are among the mechanisms implicated in the pathogenesis of diabetic cardiomyopathy. Symptoms of DCM may include, but not limited to, shortness of breath, fatigue, dizziness or fainting, arrhythmia (problem with the rate or rhythm of your heartbeat), swollen feet and ankles, and chest pain. The minimal criteria to be diagnosed with DCM include left ventricular diastolic dysfunction and/or reduced left ventricular ejection fraction (EF), pathological left ventricle hypertrophy, and interstitial fibrosis.

Non-alcoholic fatty liver disease (NAFLD) is a spectrum of chronic liver disorders, which encompass, among others nonalcoholic fatty liver (NAFL) and nonalcoholic steatohepatitis (NASH). NASH, a common cause of chronic liver disease, is defined as having at least 5% hepatic steatosis and inflammation with or without fibrosis. Over time and without treatment, NASH may progress to cirrhosis and even hepatocellular carcinoma (HCC). Moreover, NAFLD/NASH is strongly associated with obesity and type II diabetes which together affect over 50% of the US population, leading to a heavy economic burden. Unfortunately, therapeutic options for NASH remain limited, with only slight benefits observed from vitamin E or obeticholic acid treatment. While NASH is the leading cause of chronic liver disease and cirrhosis, there are currently no clinically approved therapies.

Coronavirus disease is a viral disorder caused by a coronavirus. It is generally characterized by high fever, cough, dyspnea, chills, persistent tremor, muscle pain, headache, and a viral pneumonia. In younger patients, rare inflammatory syndromes may occur, such as atypical kawasaki syndrome, toxic shock syndrome, pediatric multisystem inflammatory disease, and cytokine storm syndrome. COVID-19 and SARS (Severe Acute Respiratory Syndrome) are two severe forms of coronavirus diseases. Diabetic cardiomyopathy is diabetes complications in which ventricular remodeling in the absence of coronary atherosclerosis and hypertension results in cardiac dysfunctions, typically left ventricular dysfunction. The changes also result in myocardial hypertrophy, myocardial necrosis and fibrosis, and collagen deposition due to impaired glucose tolerance. Non-alcoholic fatty liver disease refers to fatty liver finding without excessive alcohol consumption. Fatty liver is a lipid infiltration of the hepatic parenchymal cells resulting in a yellow-colored liver. The abnormal lipid accumulation is usually in the form of triglycerides, either as a single large droplet or multiple small droplets. fatty liver is caused by an imbalance in the metabolism of fatty acids. Oxidative phosphorylation dysfunction refers to any malfunction or deficiency in the process of electron transfer through the cytochrome system liberating free energy which is transformed into high-energy phosphate bonds. It may cause a group of clinically heterogenous diseases commonly defined by lack of cellular energy due to defects of oxidative phosphorylation resulted from pathogenic mutations in the nuclear DNA, such as lactic acidosis indicating overproduction of lactate and/or decreased metabolism of lactate. Ribosomes have two main functions—decoding the message and the formation of peptide bonds. They are critical for proper protein synthesis. Ribosome dysfunctions compose a collection of disorders in which genetic abnormalities cause impaired ribosome biogenesis and function, resulting in specific clinical phenotypes. Proteasome is a multi-subunit enzyme complex that plays a central role in the regulation of proteins that control cell-cycle progression and apoptosis. Impaired proteasome can cause many diseases, including cancer and a broad array of chronic neurodegenerative diseases. Thermogenesis refers to the dissipation of energy through the production of heat and occurs in specialized tissues including brown adipose tissue and skeletal muscle. Some hormones, such as norepinephrine and leptin, may stimulate thermogenesis by activating the sympathetic nervous system. Thermogenesis dysfunction may impact normal lipid metabolism and fat regulations, which in turn cause metabolic syndrome or disorders.

Other diseases or disorders encompassed by the present disclosure include, but not limited to, a disorder related to thermogenesis, a disorder related to oxidative phosphorylation, a disorder related to proteasome dysfunction, a disorder related to ribosome dysfunction, a disorder related to retrograde endocannabinoid signaling, and a disorder related to cardiac muscle contraction.

### II. Methods of Use—Gene Profile Changes Associated with Sigma-1 Receptor Agonist Therapy

One aspect of the present disclosure encompasses a method of selecting a therapeutic agent for a subject suffering from a disease or a disorder, such as Alzheimer's disease, Parkinson's disease, Parkinson's disease with dementia, Huntington's disease, Amyotrophic lateral sclerosis, Prion disease, diabetic cardiomyopathy, non-alcoholic fatty liver disease, a coronavirus disease, a disorder related to thermogenesis, a disorder related to oxidative phosphorylation, a disorder related to proteasome dysfunction, a disorder related to ribosome dysfunction, a disorder related to retrograde endocannabinoid signaling, a disorder related to cardiac muscle contraction. It should be understood that in any of the methods disclosed herein comprise actions which may be performed in any reasonable order or sequence.

One aspect of the method comprises obtaining or having obtained a first biological sample from the subject; administering to the subject a Sigma receptor agonist for a period of time; obtaining or having obtained a second biological sample of the subject at the end of administration period; determining a gene expression profile for the first biological sample and for the second biological sample; comparing the gene expression profile of the first biological sample with that of the second biological sample to identify differentially expressed genes and overrepresented gene clusters; and selecting the Sigma receptor agonist as the therapeutic agent for the subject, if any one of the following conditions is observed: (i) the identified differentially expressed gene is a gene listed in a first gene cluster comprising COX5B, COX7B, COX8A, NDUFB6, TXN, PSMA4, CLTB, PSMB3, POLR2F, NDUFA1, PSMD13, POLR2, NDUFA2, DDIT3, CYBA, AGER, MAPA2K2, PSENEN, or NDUFB3; or a gene listed in a second gene cluster comprising COX4I1, NDUFB11, PSMA7, COX6A1, NDUFB2, PSMB1, COX6B1, NDUFB7, PSMB4, COX6C, NDUFB8, PSMB5, COX7A2, NDUFB9, PSMB6, COX7C, NDUFC1, PSMB7, CYC1, NDUFC2, PSMD8, NDUFA11, NDUFS3, RPS27A, NDUFA12, NDUFS4, SLC25A6, NDUFA13, NDUFS5, TXN2, NDUFA3, NDUFS6, UQCR10, NDUFA4, NDUFS7, UQCR11, NDUFA7, NDUFS8, UQCRH, NDUFA8, PARK7, UQCRQ, NDUFB1, PSMA2, NDUFB10, CDK5, GAPDH, HRAS, HSD17B10, or PSMA6; or (ii) the identified overrepresented gene cluster(s) overlaps with genes listed in the first gene cluster or in the second gene cluster.

Another aspect of the present disclosure encompasses a method of evaluating effectiveness of a neurodegenerative therapy for a subject in need thereof. The method comprises obtaining or having obtained a first biological sample from the subject; administering to the subject the neurodegenerative therapy for a period of time; obtaining or having obtained a second biological sample of the subject at the end of therapy period; determining a gene expression profile for the first biological sample and for the second biological sample; comparing the gene expression profile of the first biological sample with that of the second biological sample to identify differentially expressed genes and overrepresented gene clusters; and identifying the neurodegenerative therapy as effective if one of the following conditions is observed: (i) the identified differentially expressed gene is a gene listed in a first gene cluster comprising COX5B, COX7B, COX8A, NDUFB6, TXN, PSMA4, CLTB, PSMB3, POLR2F, NDUFA1, PSMD13, POLR2, NDUFA2, DDIT3, CYBA, AGER, MAPA2K2, PSENEN, or NDUFB3; or a gene listed in a second gene cluster comprising COX4I1, NDUFB11, PSMA7, COX6A1, NDUFB2, PSMB1, COX6B1, NDUFB7, PSMB4, COX6C, NDUFB8, PSMB5, COX7A2, NDUFB9, PSMB6, COX7C, NDUFC1, PSMB7, CYC1, NDUFC2, PSMD8, NDUFA11, NDUFS3, RPS27A, NDUFA12, NDUFS4, SLC25A6, NDUFA13, NDUFS5, TXN2, NDUFA3, NDUFS6, UQCR10, NDUFA4, NDUFS7, UQCR11, NDUFA7, NDUFS8, UQCRH, NDUFA8, PARK7, UQCRQ, NDUFB1, PSMA2, NDUFB10, CDK5, GAPDH, HRAS, HSD17B10, or PSMA6; or (ii) the identified overrepresented gene cluster(s) overlaps with genes listed in the first gene cluster or in the second gene cluster, wherein if neither of condition (i) or (ii) is observed, the neurodegenerative therapy is identified as ineffective for the subject.

Yet another aspect of the present disclosure encompasses a method of identifying a subject responsive to a Sigma-1 receptor agonist therapy. The method comprises obtaining or having obtained a first biological sample from the subject; administering to the subject a Sigma receptor agonist for a period of time; obtaining or having obtained a second biological sample of the subject at the end of administration period; determining a gene expression profile for the first biological sample and for the second biological sample; comparing the gene expression profile of the first biological sample with that of the second biological sample to identify differentially expressed genes and overrepresented gene clusters; identifying the subject as responsive to the Sigma-1 receptor agonist therapy, if any one of the following conditions is observed: (i) the identified differentially expressed gene is a gene listed in a first gene cluster comprising COX5B, COX7B, COX8A, NDUFB6, TXN, PSMA4, CLTB, PSMB3, POLR2F, NDUFA1, PSMD13, POLR2, NDUFA2, DDIT3, CYBA, AGER, MAPA2K2, PSENEN, or NDUFB3; or a gene listed in a second gene cluster comprising COX4I1, NDUFB11, PSMA7, COX6A1, NDUFB2, PSMB1, COX6B1, NDUFB7, PSMB4, COX6C, NDUFB8, PSMB5, COX7A2, NDUFB9, PSMB6, COX7C, NDUFC1, PSMB7, CYC1, NDUFC2, PSMD8, NDUFA11, NDUFS3, RPS27A, NDUFA12, NDUFS4, SLC25A6, NDUFA13, NDUFS5, TXN2, NDUFA3, NDUFS6, UQCR10, NDUFA4, NDUFS7, UQCR11, NDUFA7, NDUFS8, UQCRH, NDUFA8, PARK7, UQCRQ, NDUFB1, PSMA2, NDUFB10, CDK5, GAPDH, HRAS, HSD17B10, or PSMA6; (ii) the identified overrepresented gene cluster(s) overlaps with genes listed in the first gene cluster or in the second gene cluster.

In any of the methods described herein, the subject may be a human subject having or suspected of having a disorder or disease selected from Alzheimer's disease, Parkinson's disease, Parkinson's disease with dementia, Huntington's disease, Amyotrophic lateral sclerosis, Prion disease, diabetic cardiomyopathy, non-alcoholic fatty liver disease, a coronavirus disease, a disorder related to thermogenesis, a disorder related to oxidative phosphorylation, a disorder related to proteasome dysfunction, a disorder related to ribosome dysfunction, a disorder related to retrograde endocannabinoid signaling, or a disorder related to cardiac muscle contraction.

In various aspects, if the neurodegenerative therapy is identified as ineffective for a subject a follow-up action can be taken, such as switching to another neurodegenerative therapy, supplementing with another neurodegenerative therapy, adjusting dose if the neurodegenerative therapy being a drug therapy, or switching to a combined neurodegenerative therapy.

The neurodegenerative therapy can be a Sigma-1 receptor agonist therapy, or a NMDA therapy, or a cognition enhancing physical therapy. For example, a neurodegenerative therapy can be a drug therapy comprising a Sigma-1 receptor agonist, such as ANAVEX2-73 (A2-73), ANAVEX 19-144, ANAVEX1-41, AV1066, ANAVEX3-71, PRE-084, Donepezil, Fluvoxamine, Amitriptyline, L-687,384, SA-4503, Dextromethorphan, Dimethyltryptamine, (+)-pentazocine, or any of their crystal forms, enantiomers and pharmaceutically acceptable salts thereof.

As used herein, ANAVEX2-73 (A2-73) has the chemical name of tetrahydro-N,N-dimethyl-2,2-diphenyl-3-furanmethanamine hydrochloride. A2-73 free base has the chemical name of tetrahydro-N,N-dimethyl-2,2-diphenyl-3-furanmethanamine. ANAVEX 19-144 (A19-144) has the chemical name of 1-(2,2-diphenyltetrahydrofuran-3-yl)-N-methylmethanamine hydrochloride. ANAVEX 1-41 (A1-41) has the chemical name of tetrahydro-N,N-dimethyl-5,5-diphenyl-3-furanmethanamine hydrochloride. AV1066 has the chemical name of 1-(3-4(((1R,3S,5S)-adamantan-1-yl)(phenyl)methyl)propyl)-4-methylpiperazine. ANAVEX3-71 (A3-71, AF-710B) has a chemical name of 1-(2,8-dimethyl-1-thia-3,8-diazaspiro[4.5]decan-3-yl)-3-(1H-indol-3-yl)propan-1-one.

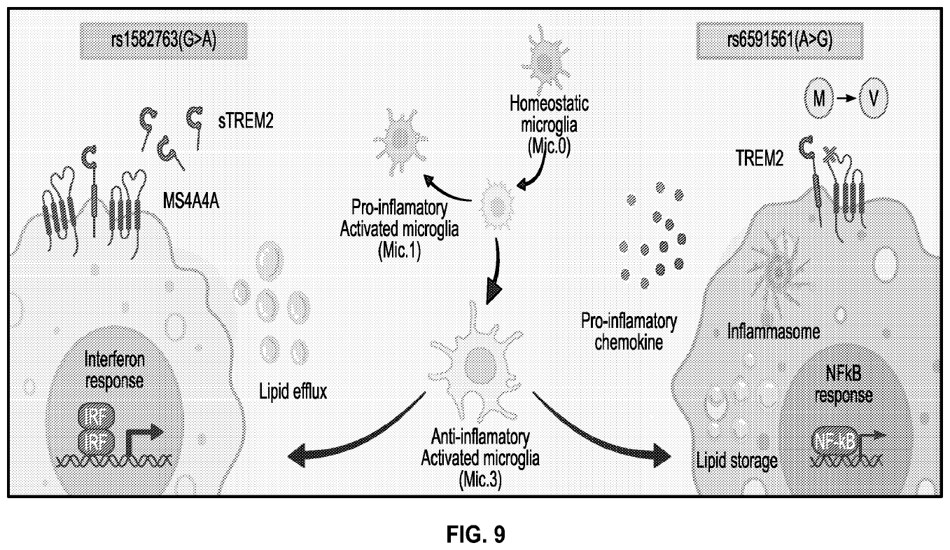

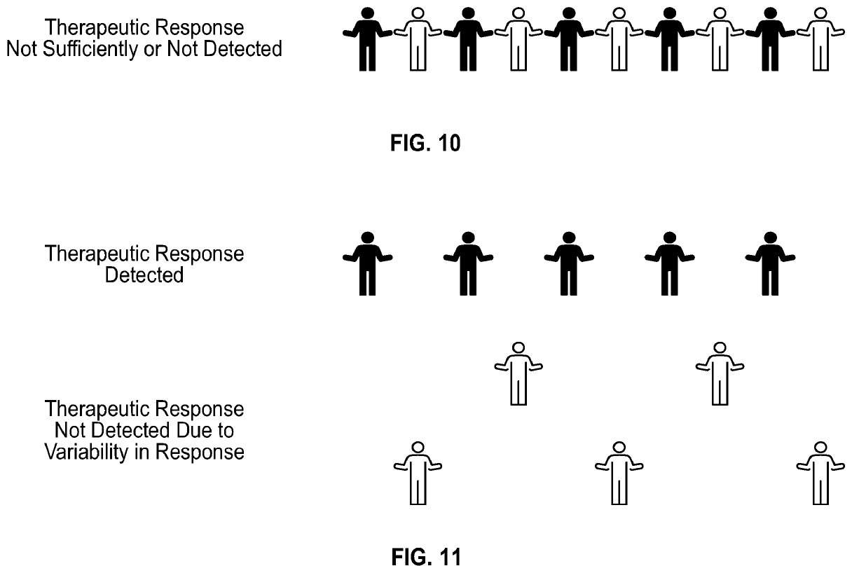

In various aspects of the methods, a neurodegenerative therapy is a A2-73 drug therapy comprising administration of A2-73 free base, A2-73 in amorphous form, A2-73 Crystal Form I, A2-73 Crystal Form II, A2-73 Crystal Form III, (−) A2-73 enantiomer, or (+) A2-73 enantiomer. The present disclosure encompasses the polymorphic forms of A2-73. The polymorphic forms may be prepared by solvent extraction or by supercritical fluid extraction (SCE). The crystal forms prepared from solvent extraction can be found in PCT/US2019/027369 (filed on Apr. 12, 2019), published as WO2019200345A1. The crystal forms prepared by SCE extraction can be found in PCT/IB2016/001181 (filed on Jul. 19, 2016), published as WO2017013498A2. The full contents of both PCT/US2019/027369 and PCT/IB2016/001181 are incorporated by reference herein. The present disclosure expressly encompasses the A2-73 Crystal Form I, A2-73 Crystal Form II and A2-73 Crystal Form III as prepared and characterized in PCT/IB2016/001181. A2-73 Crystal Form I is a crystal with XRPD peaks as shown in FIG. 1. A2-73 Crystal Form I is further characterized by scanning electron microscope (SEM) micrographs as shown in FIGS. 2-3. A2-73 Crystal Form II is a crystal with XRPD peaks as shown in FIG. 4. A2-73 Crystal Form II is also characterized by FTIR as shown in FIG. 5. The SEM micrographs of A2-73 Crystal Form II is shown in FIG. 7. A2-73 Crystal Form III is a crystal with XRPD peaks as shown in FIG. 8. A2-73 Crystal Form III is characterized by FTIR as shown in FIG. 9. A2-73 Crystal Form III is characterized by scanning electron microscope (SEM) micrograph as shown in FIG. 11. The present disclosure also encompassed the polymorphic forms of A2-73 free base. The polymorphic forms of A2-73 free base can be found in PCT/US2019/027369 (filed on Apr. 12, 2019), published as WO2019200345A1. As an example, a crystal form of A2-73 free base is the crystalline form characterized by XRPD pattern shown in FIG. 16. And such crystalline form possesses particle shapes depicted in FIG. 15. The present disclosure encompasses an enantiomer of A2-73 or a mixture of such enantiomer. The enantiomer may be an (+) or (−) enantiomer. The enantiomer refers to an optical pure or a substantial optical pure entity. The substantial optical pure entity refers to the opposite rotation entity may be presented in an amount of no more than 20%, such as at or about 0.1%, at or about 0.5%, at or about 1%, at or about 2%, at or about 5%, at or about 8%, at or about 10%, at or about 12%, at or about 15%, at or about 18%, or at or about 20%. The present disclosure expressly encompasses the (+) and (−) enantiomer disclosed and characterized in PCT/IB2016/001158 (filed on Jul. 19, 2016), published as US20180169059A1. The full contents of PCT/IB2016/001158 is expressly incorporated by reference herein.

In yet another aspect of the present disclosure, the neurodegenerative therapy or the Sigma-1 receptor agonist is administered for a period up to 14 weeks, such as about 4 weeks, about 5 weeks, about 6 weeks, about 7 weeks, about 8 weeks, about 9 weeks, about 10 weeks, about 11 weeks, about 12 weeks, about 13 weeks, or about 14 weeks. And the therapeutic agent is administered daily, such as once daily.

In yet another aspect of the present disclosure, the neurodegenerative therapy is a drug therapy comprising A2-73, for example a composition comprising A2-73 in a crystal form, and is administered in the amount of about 40 mg to about 60 mg once daily for about 6-11 weeks. For example, A2-73 is administered in about 50 mg daily for up to 11 weeks. Alternatively, A2-73 is administered daily in an escalating dose starting from about 10 mg to ending at about 50 mg once daily for 6-11 weeks.

### III. Methods Using Sigma-1 Receptor Agonist

One aspect of the present disclosure encompasses a method of determining if a subject is having or is suspected of having a disease or disorder, or assessing a risk of a subject for developing such a disease or disorder. The said disease or disorder is for example selected from Alzheimer's disease, Parkinson's disease, Parkinson's disease with dementia, Huntington's disease, Amyotrophic lateral sclerosis, Prion disease, diabetic cardiomyopathy, non-alcoholic fatty liver disease, a coronavirus disease, a disorder related to thermogenesis, a disorder related to oxidative phosphorylation, a disorder related to proteasome dysfunction, a disorder related to ribosome dysfunction, a disorder related to retrograde endocannabinoid signaling, or a disorder related to cardiac muscle contraction. In one aspect, the method comprises obtaining or having obtained a first biological sample from the subject; administering to the subject a composition of ANAVEX 2-73 for a period of time; obtaining or having obtained a second biological sample of the subject at the end of administration period; determining a gene expression profile for the first biological sample and for the second biological sample; comparing the gene expression profile of the first biological sample with that of the second biological sample to identify differentially expressed genes and overrepresented gene clusters; and identifying the subject as having a disorder or disease as listed above, or as having an increased risk of developing such disorder or disease, if any one of the following conditions is observed: (i) the identified differentially expressed gene is a gene listed in a first gene cluster comprising COX5B, COX7B, COX8A, NDUFB6, TXN, PSMA4, CLTB, PSMB3, POLR2F, NDUFA1, PSMD13, POLR2, NDUFA2, DDIT3, CYBA, AGER, MAPA2K2, PSENEN, or NDUFB3; or a gene listed in a second gene cluster comprising COX4I1, NDUFB11, PSMA7, COX6A1, NDUFB2, PSMB1, COX6B1, NDUFB7, PSMB4, COX6C, NDUFB8, PSMB5, COX7A2, NDUFB9, PSMB6, COX7C, NDUFC1, PSMB7, CYC1, NDUFC2, PSMD8, NDUFA11, NDUFS3, RPS27A, NDUFA12, NDUFS4, SLC25A6, NDUFA13, NDUFS5, TXN2, NDUFA3, NDUFS6, UQCR10, NDUFA4, NDUFS7, UQCR11, NDUFA7, NDUFS8, UQCRH, NDUFA8, PARK7, UQCRQ, NDUFB1, PSMA2, NDUFB10, CDK5, GAPDH, HRAS, HSD17B10, or PSMA6; or (ii) the identified overrepresented gene cluster(s) overlaps with genes listed in the first gene cluster or in the second gene cluster.

As in all the methods disclosed herein, the composition of A2-73 may comprise A2-73 free base, A2-73 amorphous form, A2-73 Crystal Form I, A2-73 Crystal Form II, A2-73 Crystal Form III, (−) A2-73 enantiomer, (+) A2-73 enantiomer, or any combination thereof.

In any of the methods, a composition comprising A2-73 can be administered to the subject for a period of up to 14 weeks, for example 2-3 weeks, about 4 weeks, about 5 weeks, about 6 weeks, about 7 weeks, about 8 weeks, about 9 weeks, about 10 weeks, about 11 weeks, about 12 weeks, about 13 weeks, or about 14 weeks. The therapeutic agent is for example administered daily, such as once daily.

In any of the methods, the composition of A2-73 may comprise a crystal of A2-73 or A2-73 free base, and is administered in the amount of about 40 mg to about 60 mg once daily for about 6-11 weeks. For example, the crystal of A2-73 or A2-73 free base is administered in about 50 mg daily for up to 11 weeks. Or A2-73 or A2-73 free base is administered daily in an escalating dose starting from about 10 mg to ending at about 50 mg once daily for 6-11 weeks.

In yet another one aspect of the present disclosure, the method comprises a biological sample. A biological sample may comprise a solid, a semi-solid, a semi-fluid, a fluid, a tissue, or other material collected from a subject. A biological sample may be a fluid biological sample. A fluid biological sample may include fluid and some or all of cells, particles, crystals, and other components in the fluid. Examples of biological samples include but are not limited to, blood, serum, plasma, bone marrow, a nasal swab, a nasopharyngeal wash, saliva, urine, gastric fluid, spinal fluid, tears, stool, mucus, sweat, earwax, oil, a glandular secretion, cerebral spinal fluid, tissue, semen, vaginal fluid, interstitial fluids derived from tumorous tissue, ocular fluids, spinal fluid, a throat swab, breath, hair, finger nails, skin, biopsy, placental fluid, amniotic fluid, cord blood, lymphatic fluids, cavity fluids, sputum, pus, microbiota, meconium, breast milk and/or other secretions or excretions. Biological samples may include nasopharyngeal wash, or other fluid obtained by washing a body cavity or surface of a subject, or by washing a swab following application of the swab to a body cavity or surface of a subject. Nasal swabs, throat swabs, stool samples, hair, finger nail, ear wax, breath, and other solid, semi-solid, or gaseous samples may be processed in an extraction buffer, e.g., for a fixed or variable amount of time. Examples of tissue samples of the subject may include but are not limited to, connective tissue, muscle tissue, nervous tissue, epithelial tissue, cartilage, cancerous sample, or bone. The sample may be obtained from a human or animal. The sample may be obtained from a vertebrate, e.g., a bird, fish, or mammal, such as a rat, a mouse, a pig, an ape, another primate (including humans), a farm animal, a sport animal, or a pet. The sample may be obtained from a living or dead subject. The sample may be obtained fresh from a subject or may have undergone some form of pre-processing, storage, or transport. The terms “blood” and “whole blood” refer to blood as it exists within an animal and as directly obtained from a subject in a blood sample. Blood contains red blood cells, white blood cells, proteins such as albumin, globulins, and clotting factors, salts, water, and other constituents. A blood sample is a fluid biological sample. In one aspect, a biological sample refers to a fluid sample containing suspended cells, cellular material, fungi, antigens, biological factors, immunogens, proteins, and other material of biological origin generally having a size substantially larger than the size of the molecules that comprise the fluid. Biological factors refer to compounds made by living organisms or attached to living organisms that have biological or physiological activities. Biological factors include but are not limited to biological markers, antibodies, cytokines, growth factors, and other peptides, proteins, lipids and carbohydrates produced biologically. terms “plasma” and “blood plasma” refer to the liquid portion of blood (e.g., a blood sample) that remains after the removal of blood cells. Red blood cells and white blood cells may be removed by centrifugation of a blood sample, leaving plasma above the pelleted cells in the bottom of the centrifuge tube. Plasma retains blood clotting factors, and is obtained from anti-coagulated blood samples. A sample of plasma is a fluid biological sample. The terms “serum” and “blood serum” refer to the liquid portion of blood that remains after blood is allowed to clot, and the clot is removed. Serum differs from plasma in that serum lacks clotting factors: since clotting requires fibrin, thrombin, and other proteins, which form and remain part of a blood clot, serum lacks these proteins while plasma contains them. A sample of blood serum is a fluid biological sample. The present disclosure comprises a first biological sample and/or a second biological sample. The first and the second biological sample can be the same type of biological sample, but taken at different time. The first and the second biological sample may be a different type of biological sample taken from the same subject. The first and the second biological sample may be different type of biological sample taken at different time from the same subject. The first and the second biological sample may be the same type of biological sample taken at the same time, but from different subjects. The first and the second biological sample each is independently any biological sample recited above. In one aspect, the first and the second biological sample each independently, comprises a blood serum sample, a plasma sample, or a biological sample suitable for transcriptomic analysis. Transcriptome analysis is the study of the transcriptome, of the complete set of RNA transcripts that are produced by the genome, under specific circumstances or in a specific cell, often using high-throughput methods. The transcriptomic analysis is useful in identifying the functions of genes, and/or identification of pathways that respond to or ameliorate environmental stresses. RNA-Seq can also identify disease-associated gene fusions, single nucleotide polymorphisms and even allele-specific expression. The present disclosure expressly incorporate any biological sample that are suitable and/or capable of being used for transcriptomic analysis.

### IV. Kits and Medical Use

One aspect of the present disclosure encompasses a kit for a medical use according to any of the monitoring, evaluating, or therapy selection methods disclosed herein. A kit for such uses includes one or more containers for receiving and holding biological samples that may be taken at different points in time, and reagents for carrying out gene expression analysis. For example, the kit may have a first container for receiving a first biological sample as used in the disclosed methods; a second container for receiving a second biological sample as used in the disclosed methods; and additional one or more containers for receiving and/or storing reagent(s) useful for sequencing or measuring a gene expression. Additional containers may be included to receive and store composition(s) in a safe, stable and durable way. The composition may comprise a Sigma-1 receptor agonist, such as a composition comprising A2-73 or A2-73 free base. The kit can include an instruction in hard copy printed from or in a computer readable form, which explains the use of the kit components to perform any of the methods described herein. The instruction may be written with the medical practitioner and/or the patient as the intended reader.

In one aspect of the present disclosure, the kit may be used for various purposes, such as selection of a therapeutic agent for a subject, evaluation effectiveness of a neurodegenerative therapy, identification of a subject responsive to a Sigma-1 receptor agonist therapy, or determining if a subject is having, is suspected of having, or having an increased risk for developing a disorder or a disease. Such disorder or disease may include Alzheimer's disease, Parkinson's disease, Parkinson's disease with dementia, Huntington's disease, Amyotrophic lateral sclerosis, Prion disease, diabetic cardiomyopathy, non-alcoholic fatty liver disease, a coronavirus disease, a disorder related to thermogenesis, a disorder related to oxidative phosphorylation, a disorder related to proteasome dysfunction, a disorder related to ribosome dysfunction, a disorder related to retrograde endocannabinoid signaling, or a disorder related to cardiac muscle contraction.

### V. Methods of Treatment

One aspect of the present disclosure encompasses a method of treating a disorder or a disease in a subject by administering a therapeutically effective amount of a Sigma-1 receptor agonist. The disorder or disease comprises Alzheimer's disease, Parkinson's disease, Parkinson's disease with dementia, Huntington's disease, Amyotrophic lateral sclerosis, Prion disease, diabetic cardiomyopathy, non-alcoholic fatty liver disease, a coronavirus disease, a disorder related to thermogenesis, a disorder related to oxidative phosphorylation, a disorder related to proteasome dysfunction, a disorder related to ribosome dysfunction, a disorder related to retrograde endocannabinoid signaling, a disorder related to cardiac muscle contraction, or any combination thereof. The Sigma-1 receptor agonist may comprise ANAVEX2-73 (A2-73) in any form, ANAVEX 19-144, ANAVEX1-41, AV1066, ANAVEX3-71, PRE-084, Donepezil, Fluvoxamine, Amitriptyline, L-687,384, SA-4503, Dextromethorphan, Dimethyltryptamine, (+)-pentazocine, or any of their crystal forms, enantiomers and pharmaceutically acceptable salts thereof. In one aspect, the treatment is for Parkinson's Disease, Parkinson's disease with dementia, or for treating Prion Disease.

In any of these treatment methods, the Sigma-1 receptor agonist may be A2-73 in the forms of A2-73 free base, A2-73 amorphous form, A2-73 Crystal Form I, A2-73 Crystal Form II, A2-73 Crystal Form III, (−) A2-73 enantiomer, (+) A2-73 enantiomer, or any combination thereof. And the therapeutically effective amount of A2-73 comprises A2-73 in an amount of 0.5-100 mg per day, such 10-50 mg per day. A2-73, the therapeutically effective amount of A2-73 can range from about 0.5 mg to about 20 mg, about 1 mg to about 60 mg, about 30 mg to about 50 mg, or about 3 mg to about 5 mg. The therapeutically effective amount of A2-73 can range from about 0.5 mg/day to about 100 mg/day, from about 1 to about 60 mg/day, from about 20 to about 50 mg/day, from about 20 to about 30 mg/day, or from about 15 to about 25 mg/day. Administering the anti-neurodegenerative effective amount of A2-73 can provide blood levels of about 10 ng/ml, about 12 ng/ml of A2-73.

A2-73 can be administered to the subject daily or more than once daily. Further, A2-73 can be administered every 2, 3, 4, 5, 6, 7, 14, or every 30 days. A2-73 can be administered over a period ranging from about 1 day to about 1 year, from about 1 day to about 1 week, from about 3 days to about 1 month, from about 2 weeks to about 6 months, or from about 2 months to about 4 months. A2-73 can also be administered over a period of about 1 day, about 7 days, about 30 days, about 60 days, about 120 days, or about 180 days or more. In some aspects, A2-73 is administered over a period of about 6 weeks, 8 weeks, 10 weeks, 11 weeks, 12 weeks, 13 weeks, 14 weeks, 57 weeks, about 148 weeks, about 208 weeks, indefinitely, or until resolution of the condition being treated.

Other methods of administering A2-73 can be found in, e.g., U.S. Pat. No. 9,750,746, U.S. Patent Publication No. 20170360798, U.S. Patent Publication No. 20190022052, U.S. Patent Publication No. 20180360796, U.S. Patent Publication No. 20180169059, U.S. Patent Publication No. 20180177756, U.S. Patent Publication No. 20180169060, and U.S. Patent Publication No. 20190117615, the disclosures of which are incorporated herein in their entirety.

### VI. Biological Samples and Analysis Through Modeling

Any biological samples suitable for transcriptome or gene analysis can be used in the present disclosure. Non-limiting examples of suitable biological samples include fluid samples, biopsy samples, skin samples, and hair samples. Fluid samples may include blood, serum, plasma, saliva, tears, and lymph. In some aspects the biological sample is a blood sample, a serum sample, a plasma sample or any transcroptomic sample. Methods of collecting a biological sample from a subject are well known in the art.

Gene expression measurement includes both DNA and RNA measurements. They may be accomplished by a variety of methods including northern blotting, quantitative real-time PCR (qRT-PCR), nucleic acid microarrays, Luminex microspheres, and nuclease protection assay. Methods of determining a substantially different level of RNA expression are known in the art, and include distribution analysis. In some aspects, a substantially different level of RNA expression is determined by normalizing RNA expression values as Transcripts Per Kilobase Million (TPM).

Over the last decade, there is an avalanche of biomedical data generation and a parallel expansion in computational capabilities to analyze and make sense of these data. Starting with genome sequencing (such as omics) and widely employed deep sequencing technologies, these trends have now taken hold in all omics disciplines and increasingly call for multi-omics integration as well as data interpretation by artificial intelligence technologies. Artificial intelligence (AI) aided-data modeling (AI Modeling) has made the interpretation of large omics data sets feasible and meaningful. Use of omics and AI Modeling in biomarker discovery can aid clinicians by identifying markers of risk for developing a certain disease, monitoring care, determining prognosis, and developing druggable targets. Combined, AI Modeling has the power to improve patient care, and facilitates the development of precision medicine. In the present disclosure, the AI Modeling not only considers omics data, but also a broad range of other subjects' data. Such data include, but not limited to, subject's clinical diagnosis, co-morbidity, co-medication, age, weight, height, and gender. The AI Modeling used in the present disclosure also include trial data, such as doses received, dosing time points, endpoint assessment, and behavior impacts, such as sleep or cognition impacts. Thorough data collection and advanced algorithm, the AI Modeling in the present disclosure is able to generate and analyze over 24 million relationships and concludes with biomarkers with convincing confidence and p values.

### VII. Pharmaceutical Compositions

One aspect of the disclosure encompasses a pharmaceutical composition comprising a neurodegenerative agent and/or a Sigma-1 receptor agonist. A pharmaceutical composition comprises a therapeutically effective amount of an active pharmaceutical ingredient, and any pharmaceutically acceptable salt thereof.

Pharmaceutically acceptable salts, include, without limitation, acetate, aspartate, benzoate, bitartrate, citrate, formate, gluconate, glucuronate, glutamate, fumarate, hydrochloride, hydrobromide, hydroiodide, hypophosphite, isobutyrate, isocitrate, lactate, malate, maleate, meconate, methylbromide, methanesulfonate, monohydrate, mucate, nitrate, oxalate, phenylpropionate, phosphate, phthalate, propionate, pyruvate, salicylate, stearate, succinate, sulfate, tannate, tartrate, terephthalate, valerate, and the like.

When the active pharmaceutical ingredient is A2-73, a composition may comprise from about 1 mg to about 50 g, from about 0.1 to about 5 g, from about 0.5 g to about 3 g, from about 1 mg to about 55 mg, from about 40 mg to about 60 mg, from about 80 mg to about 120 mg, from about 180 mg to about 220 mg, from about 0.1 g to about 5 g, or from about 0.5 g to about 3 g of A2-73. Formulations comprising A2-73 can be found in, e.g., U.S. Pat. No. 9,750,746, U.S. Patent Publication No. 20170360798, U.S. Patent Publication No. 20190022052, U.S. Patent Publication No. 20180360796, U.S. Patent Publication No. 20180169059, U.S. Patent Publication No. 20180177756, U.S. Patent Publication No. 20180169060, and U.S. Patent Publication No. 20190117615, the disclosures of which are incorporated herein in their entirety.

The active pharmaceutical ingredient can be formulated and administered to a subject by several different means. For instance, a composition can generally be administered parenteraly, intraperitoneally, intravascularly, transdermally, subcutaneously, or intrapulmonarily in dosage unit formulations containing conventional nontoxic pharmaceutically acceptable adjuvants, carriers, excipients, and vehicles as desired. The term parenteral as used herein includes subcutaneous, intravenous, intramuscular, intrathecal, or intrasternal injection, or infusion techniques. Formulation of pharmaceutical compositions is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

A pharmaceutical composition also comprises one or more pharmaceutically acceptable excipients. Non-limiting examples of excipients include chemical enhancers, humectants, pressure sensitive adhesives, antioxidants, solubilizers, thickening agents, plasticizers, adjuvants, carriers, excipients, vehicles, coatings, and any combinations thereof. One or more excipients can be selected for oral, transdermal, parenteral, intraperitoneal, intravascular, subcutaneous, by inhalation spray, rectal, or intrapulmonary administration.

The active pharmaceutical ingredient can in general be formulated for improving patient compliance, preventing a subject from removing the drug-delivery device. For instance, Sigma-1 receptor agonists could be formulated for improved patient compliance and preventing removal of a drug-delivery device by providing formulations for extended delivery. Extended delivery can range for periods ranging from more than one day, to months. This may be especially relevant for patients with compromised cognitive and/or motor-control abilities. Extended delivery for periods can range from about 1 day to about 1 year, from about 1 day to about 1 week, from about 3 days to about 1 month, from about 2 weeks to about 6 months, or from about 2 months to about 4 months.

Extended release formulations could be used for substantially continuous delivery of drug at a preselected rate. For example, for crystalline A2-73, the drug can be delivered at a rate of from about 1 mg to about 100 mg/day, from about 40 to about 60 mg/day, or from about 10 to about 30 mg/day. Appropriate amounts of crystalline A2-73 can be readily determined by the ordinarily skilled artisan based upon, for example, the intended duration of administration of the drug by the extended release formulation, the delivery mechanism, the particular formulation, and the relative potency of the drug among other factors.

### Binders

Non-limiting examples of binders suitable for the formulations of various aspects include starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C12-C18 fatty acid alcohols, polyethylene glycol, polyols, saccharides, oligosaccharides, polypeptides, oligopeptides, and combinations thereof. The polypeptide may be any arrangement of amino acids ranging from about 100 to about 300,000 Daltons.

The binder can be introduced into the mixture to be granulated in a solid form, including but not limited to a crystal, a particle, a powder, or any other finely divided solid form known in the art. Alternatively, the binder can be dissolved or suspended in a solvent and sprayed onto the mixture in a granulation device as a binder fluid during granulation.

### Diluents

Non-limiting examples of diluents (also referred to as “fillers” or “thinners”) include carbohydrates, inorganic compounds, and biocompatible polymers, such as polyvinylpyrrolidone (PVP). Other non-limiting examples of diluents include dibasic calcium sulfate, tribasic calcium sulfate, starch, calcium carbonate, magnesium carbonate, microcrystalline cellulose, dibasic calcium phosphate, tribasic calcium phosphate, magnesium carbonate, magnesium oxide, calcium silicate, talc, modified starches, saccharides such as sucrose, dextrose, lactose, microcrystalline cellulose, fructose, xylitol, and sorbitol, polyhydric alcohols; starches; pre-manufactured direct compression diluents; and mixtures of any of the foregoing.

### Disintegrants

Disintegrents can be effervescent or non-effervescent. Non-limiting examples of non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Suitable effervescent disintegrants include but are not limited to sodium bicarbonate in combination with citric acid, and sodium bicarbonate in combination with tartaric acid.

### Preservatives

Non-limiting examples of preservatives include, but are not limited to, ascorbic acid and its salts, ascorbyl palmitate, ascorbyl stearate, anoxomer, N-acetylcysteine, benzyl isothiocyanate, m-aminobenzoic acid, o-aminobenzoic acid, p-aminobenzoic acid (PABA), butylated hydroxyanisole (BHA), butylated hydroxytoluene (BHT), caffeic acid, canthaxantin, alpha-carotene, beta-carotene, beta-caraotene, beta-apo-carotenoic acid, carnosol, carvacrol, catechins, cetyl gallate, chlorogenic acid, citric acid and its salts, clove extract, coffee bean extract, p-coumaric acid, 3,4-dihydroxybenzoic acid, N,N′-diphenyl-p-phenylenediamine (DPPD), dilauryl thiodipropionate, distearyl thiodipropionate, 2,6-di-tert-butylphenol, dodecyl gallate, edetic acid, ellagic acid, erythorbic acid, sodium erythorbate, esculetin, esculin, 6-ethoxy-1,2-dihydro-2,2,4-trimethylquinoline, ethyl gallate, ethyl maltol, ethylenediaminetetraacetic acid (EDTA), eucalyptus extract, eugenol, ferulic acid, flavonoids (e.g., catechin, epicatechin, epicatechin gallate, epigallocatechin (EGC), epigallocatechin gallate (EGCG), polyphenol epigallocatechin-3-gallate), flavones (e.g., apigenin, chrysin, luteolin), flavonols (e.g., datiscetin, myricetin, daemfero), flavanones, fraxetin, fumaric acid, gallic acid, gentian extract, gluconic acid, glycine, gum guaiacum, hesperetin, alpha-hydroxybenzyl phosphinic acid, hydroxycinammic acid, hydroxyglutaric acid, hydroquinone, N-hydroxysuccinic acid, hydroxytryrosol, hydroxyurea, rice bran extract, lactic acid and its salts, lecithin, lecithin citrate; R-alpha-lipoic acid, lutein, lycopene, malic acid, maltol, 5-methoxy tryptamine, methyl gallate, monoglyceride citrate; monoisopropyl citrate; morin, beta-naphthoflavone, nordihydroguaiaretic acid (NDGA), octyl gallate, oxalic acid, palmityl citrate, phenothiazine, phosphatidylcholine, phosphoric acid, phosphates, phytic acid, phytylubichromel, pimento extract, propyl gallate, polyphosphates, quercetin, trans-resveratrol, rosemary extract, rosmarinic acid, sage extract, sesamol, silymarin, sinapic acid, succinic acid, stearyl citrate, syringic acid, tartaric acid, thymol, tocopherols (i.e., alpha-, beta-, gamma- and delta-tocopherol), tocotrienols (i.e., alpha-, beta-, gamma- and delta-tocotrienols), tyrosol, vanilic acid, 2,6-di-tert-butyl-4-hydroxymethylphenol (i.e., Ionox 100), 2,4-(tris-3′,5′-bi-tert-butyl-4′-hydroxybenzyl)-mesitylene (i.e., Ionox 330), 2,4,5-trihydroxybutyrophenone, ubiquinone, tertiary butyl hydroquinone (TBHQ), thiodipropionic acid, trihydroxy butyrophenone, tryptamine, tyramine, uric acid, vitamin K and derivates, vitamin Q10, wheat germ oil, zeaxanthin, or combinations thereof.

### Flavor-Modifying Agents

Suitable flavor-modifying agents include flavorants, taste-masking agents, sweeteners, and the like. Flavorants include, but are not limited to, synthetic flavor oils and flavoring aromatics and/or natural oils, extracts from plants, leaves, flowers, fruits, and combinations thereof. Other non-limiting examples of flavors include cinnamon oils, oil of wintergreen, peppermint oils, clover oil, hay oil, anise oil, eucalyptus, vanilla, citrus oils such as lemon oil, orange oil, grape and grapefruit oil, fruit essences including apple, peach, pear, strawberry, raspberry, cherry, plum, pineapple, and apricot.

Taste-masking agents include but are not limited to cellulose hydroxypropyl ethers (HPC) such as Klucel®, Nisswo HPC and PrimaFlo HP22; low-substituted hydroxypropyl ethers (L-HPC); cellulose hydroxypropyl methyl ethers (HPMC) such as Seppifilm-LC, Pharmacoat®, Metolose SR, Opadry YS, PrimaFlo, MP3295A, Benecel MP824, and Benecel MP843; methylcellulose polymers such as Methocel® and Metolose®; Ethylcelluloses (EC) and mixtures thereof such as E461, Ethocel®, Aqualon®-EC, Surelease; Polyvinyl alcohol (PVA) such as Opadry AMB; hydroxyethylcelluloses such as Natrosol®; carboxymethylcelluloses and salts of carboxymethylcelluloses (CMC) such as Aualon®-CMC; polyvinyl alcohol and polyethylene glycol co-polymers such as Kollicoat IR®; monoglycerides (Myverol), triglycerides (KLX), polyethylene glycols, modified food starch, acrylic polymers and mixtures of acrylic polymers with cellulose ethers such as Eudragit® EPO, Eudragit® RD100, and Eudragit® E100; cellulose acetate phthalate; sepifilms such as mixtures of HPMC and stearic acid, cyclodextrins, and mixtures of these materials. In other aspects, additional taste-masking agents contemplated are those described in U.S. Pat. Nos. 4,851,226; 5,075,114; and 5,876,759, each of which is hereby incorporated by reference in its entirety.

Non-limiting examples of sweeteners include glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia rebaudiana (Stevioside); chloro derivatives of sucrose such as sucralose; sugar alcohols such as sorbitol, mannitol, sylitol, hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof.

### Lubricants and Glidants

The lubricant compositions may be utilized to lubricate ingredients that form a pharmaceutical composition. As a glidant, the lubricant facilitates removal of solid dosage forms during the manufacturing process. Non-limiting examples of lubricants and glidants include magnesium stearate, calcium stearate, zinc stearate, hydrogenated vegetable oils, sterotex, polyoxyethylene monostearate, talc, polyethylene glycol, sodium benzoate, sodium lauryl sulfate, magnesium lauryl sulfate, and light mineral oil. The pharmaceutical composition will generally comprise from about 0.01% to about 10% by weight of a lubricant. In some aspects, the pharmaceutical composition will comprise from about 0.1% to about 5% by weight of a lubricant. In a further aspect, the pharmaceutical composition will comprise from about 0.5% to about 2% by weight of a lubricant.

### Dispersants

Dispersants may include but are not limited to starch, alginic acid, polyvinylpyrrolidones, guar gum, kaolin, bentonite, purified wood cellulose, sodium starch glycolate, isoamorphous silicate, and microcrystalline cellulose as high hydrophilic-lipophilic balance (HLB) emulsifier surfactants.

### Colorants

Depending upon the aspect of the disclosure, it may be desirable to include a coloring agent. Suitable color additives include but are not limited to food, drug and cosmetic colors (FD&C), drug and cosmetic colors (D&C), or external drug and cosmetic colors (Ext. D&C). These colors or dyes, along with their corresponding lakes, and certain natural and derived colorants, may be suitable for use in various aspects of the disclosure.

### PH Modifiers

Non-limiting examples of pH modifiers include citric acid, acetic acid, tartaric acid, malic acid, fumaric acid, lactic acid, phosphoric acid, sorbic acid, benzoic acid, sodium carbonate and sodium bicarbonate.

### Chelating Agents

A chelating agent may be included as an excipient to immobilize oxidative groups, including but not limited to metal ions, in order to inhibit the oxidative degradation of the morphinan by these oxidative groups. Non-limiting examples of chelating agents include lysine, methionine, glycine, gluconate, polysaccharides, glutamate, aspartate, and disodium ethylenediaminetetraacetate (Na2EDTA).

### Antimicrobial Agents

An antimicrobial agent may be included as an excipient to minimize the degradation of the compound according to this disclosure by microbial agents, including but not limited to bacteria and fungi. Non-limiting examples of antimicrobials include parabens, chlorobutanol, phenol, calcium propionate, sodium nitrate, sodium nitrite, Na2EDTA, and sulfites including but not limited to sulfur dioxide, sodium bisulfite, and potassium hydrogen sulfite.

### Release-Controlling Polymers

Release-controlling polymers may be included in the various aspects of the solid dosage pharmaceutical compositions incorporating compounds according to this disclosure. In one aspect, the release-controlling polymers may be used as a tablet coating. In other aspects, including but not limited to bilayer tablets, a release-controlling polymer may be mixed with the granules and other excipients prior to the formation of a tablet by a known process including but not limited to compression in a tablet mold. Suitable release-controlling polymers include but are not limited to hydrophilic polymers and hydrophobic polymers.

Suitable hydrophilic release-controlling polymers include, but are not limited to, cellulose acetate, cellulose diacetate, cellulose triacetate, cellulose ethers, hydroxyethyl cellulose, hydroxypropyl cellulose, hydroxypropyl methylcellulose, microcrystalline cellulose, nitrocellulose, crosslinked starch, agar, casein, chitin, collagen, gelatin, maltose, mannitol, maltodextrin, pectin, pullulan, sorbitol, xylitol, polysaccharides, ammonia alginate, sodium alginate, calcium alginate, potassium alginate, propylene glycol alginate, alginate sodium carmellose, calcium carmellose, carrageenan, fucoidan, furcellaran, arabic gum, carrageens gum, ghafti gum, guar gum, karaya gum, locust bean gum, okra gum, tragacanth gum, scleroglucan gum, xanthan gum, hypnea, laminaran, acrylic polymers, acrylate polymers, carboxyvinyl polymers, copolymers of maleic anhydride and styrene, copolymers of maleic anhydride and ethylene, copolymers of maleic anhydride propylene or copolymers of maleic anhydride isobutylene), crosslinked polyvinyl alcohol and poly N-vinyl-2-pyrrolidone, diesters of polyglucan, polyacrylamides, polyacrylic acid, polyamides, polyethylene glycols, polyethylene oxides, poly(hydroxyalkyl methacrylate), polyvinyl acetate, polyvinyl alcohol, polyvinyl chloride, polystyrenes, polyvinylpyrrolidone, anionic and cationic hydrogels, and combinations thereof.

### Coatings

A solid dosage comprising a compound according to this disclosure may comprise a coating, wherein such a coating may control release of the compound, act as a moisture barrier, or buffer or modify pH. A “control releasing coat” or “controlled release coat” as used herein is defined to mean a functional coat which can for example comprise at least one pH independent polymer, pH dependent polymer (for example enteric or reverse enteric type polymers), soluble polymer, insoluble polymer, lipids, lipidic materials, or combinations thereof. The coating, when applied onto a dosage form, may slow (for example when applied to a normal release matrix dosage form), further slow (for example when applied to a controlled release matrix dosage form) or modify the rate of release of a compound according to this disclosure when applied to an uncoated dosage form. For example, the control releasing coat can be designed such that when the control releasing coat is applied to a dosage form, the dosage form in conjunction with the control releasing coat can exhibit the release of the compound according to this disclosure, such as a “modified-release”, “controlled-release”, “sustained-release”, “extended-release”, “delayed-release”, “prolonged-release,” or combinations thereof. The “control releasing coat” may optionally comprise additional materials that may alter the functionality of the control releasing coat.

The term “moisture barrier” as used herein is one which impedes or retards the absorption of moisture. Compounds according to this disclosure may be hygroscopic and, as such, may be susceptible to decomposition over time under highly humid conditions. The proportion of the components of the moisture barrier and the amount of the moisture barrier optionally applied onto the control-releasing coating or onto the core are typically such that the moisture barrier does not fall within the USP definition and requirement for an enteric coat. Suitably, the moisture barrier may comprise an enteric and/or acrylic polymer, suitably an acrylic polymer, optionally a plasticizer, and a permeation enhancer. The permeation enhancer is a hydrophilic substance, which allows water to enter without physical disruption of the coating. The moisture barrier may additionally comprise other conventional inert excipients, which may improve processing of an extended-release formulation.

Coating and matrix materials which may be used in accordance with the present disclosure are those known in the art for use in controlled-release formulations, such as synthetic polymers of the polyvinyl type, e.g., polyvinylchloride, polyvinylacetate and copolymers thereof, polyvinylalcohol, and polyvinylpyrrolidone; synthetic polymers of the polyethylene type, e.g., polyethylene and polystyrene; acrylic acid polymers; biopolymers or modified biopolymers, such as cellulosic polymers, shellac and gelatin; fats, oils, higher fatty acids and higher alcohols (i.e., acids and alcohols containing alkyl chains of at least 10 carbon atoms), for example aluminum monostearate, cetylalcohol, hydrogenated beef tallow, hydrogenated castor oil, 12-hydroxystearl alcohol, glyceryl mono- or dipalmitate; glyceryl mono-, di- or tristearate; myristyl alcohol, stearic acid, stearyl alcohol, and polyethyleneglycols; waxes; sugars and sugar alcohols.

The pH-buffering properties of a coating may be strengthened by introducing into the coating substances chosen from a group of compounds usually used in antacid formulations, for example magnesium oxide, hydroxide or carbonate, aluminum or calcium hydroxide, carbonate or silicate; composite aluminum/magnesium compounds, for example Al2O3·6MgO·CO2·12H2O, (Mg6Al2(OH)16CO3·4H2O), MgO·Al2O3·2SiO2·nH2O, aluminum bicarbonate coprecipitate or similar compounds; or other pharmaceutically acceptable pH-buffering compounds, for example the sodium, potassium, calcium, magnesium and aluminum salts of phosphoric, carbonic, citric or other suitable, weak, inorganic or organic acids; or suitable organic bases, including basic amino acids; and salts or combinations thereof.

A pH-dependent coating serves to release the drug in desired areas of the gastrointestinal (GI) tract, e.g., the stomach or small intestine. When a pH-independent coating is desired, the coating is designed to achieve optimal release regardless of pH-changes in the environmental fluid, e.g., the GI tract. When the coating is formulated to release a compound according to this disclosure in the intestines (especially the upper small intestines), the coating is often called an “enteric coating”. A pH-dependent coating may include, but is not limited to, acrylic acid polymers and copolymers, for example polymers formed from acrylic acid, methacrylic acid, methyl acrylate, ammonio methylacrylate, ethyl acrylate, methyl methacrylate and/or ethyl methacrylate (e.g., Eudragit™); cellulosic polymers such as hydroxypropyl cellulose, hydroxyethyl cellulose, hydroxypropyl methyl cellulose, methyl cellulose, ethyl cellulose, cellulose acetate, cellulose acetate phthalate (CAP), cellulose acetate trimellitate, hydroxypropylmethyl cellulose phthalate, hydroxypropylmethyl cellulose succinate and carboxymethylcellulose sodium; shellac (purified lac); vinyl polymers and copolymers such as polyvinyl pyrrolidone, polyvinyl acetate, polyvinylacetate phthalate (PVAP), vinylacetate crotonic acid copolymer, and ethylene-vinyl acetate copolymers; zein; and salts and combinations thereof. Oral tables and capsules typically have coatings.

### Definitions

Unless defined otherwise, all technical and scientific terms used herein have the meaning commonly understood by a person skilled in the art to which present disclosure belongs. The following references provide one of skill with a general definition of many of the terms used in present disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above-described cells and methods without departing from the scope of the present disclosure, it is intended that all matter contained in the above description and in the examples given below, shall be interpreted as illustrative and not in a limiting sense.

The term “comprising” means “including, but not necessarily limited to”; it specifically indicates open-ended inclusion or membership in a so-described combination, group, series and the like. The terms “comprising” and “including” as used herein are inclusive and/or open-ended and do not exclude additional, unrecited elements or method processes. The term “consisting essentially of” is more limiting than “comprising” but not as restrictive as “consisting of.” Specifically, the term “consisting essentially of” limits membership to the specified materials or items and those that do not materially affect the essential characteristics of the present disclosure.

As used herein, the term “gene” means a segment of DNA that contains all the information for the regulated biosynthesis of an RNA product, including promoters, exons, introns, and other untranslated regions that control expression. As used herein, “expression” includes but is not limited to one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function. As used herein, the term “differentially expressed gene” means expression levels of a gene in two experimental conditions or in two samples possess statistically significant difference or change. As used herein, the terms “overrepresented” genes or gene clusters means genes from a pre-defined set are present more than expected.

As used herein, the term “transcriptome analysis” means to characterize transcriptional activity (coding and non-coding), focus on a subset of relevant target genes and transcripts, or profile thousands of genes at once to create a genetic profile. As used herein, the term “mutant” means any heritable variation from the wild-type that is the result of a mutation, e.g., single nucleotide polymorphisms (“SNP”) and insertions/deletions. The term “mutant” is used interchangeably with the terms “marker”, “biomarker”, and “target” throughout the specification.

As used herein, the term “polynucleotide” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

As used herein, the term “polypeptide” means any polypeptide comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well-known in the art. Such modifications are well described in basic texts and in more detailed monographs, as well as in a voluminous research literature.

As used herein, the terms “disease”, “disorder” or “dysfunction” are used interchangeably in the present disclosure. They refer to any condition, disorder or disease manifested as one or more physiological, physical and/or psychological symptoms or dysfunctions for which treatment is desirable, and includes previously and newly identified diseases, disorders or dysfunctions on any organs, tissues or biological activities. As used herein, the term “medical use” is any use or means related to restore, remedy, or preserve health or well being of a subject.

As used herein, the term “subject” means that preferably the subject is a mammal, such as a human, but can also be an animal, e.g., domestic animals (e.g., dogs, cats and the like), farm animals (e.g., cows, sheep, pigs, horses and the like) and laboratory animals (e.g., cynomolgus monkey, rats, mice, guinea pigs and the like).

As used herein, the administration of an agent or drug to a subject or patient includes self-administration and the administration by another. It is also to be appreciated that the various modes of treatment or prevention of medical conditions as described are intended to mean “substantial”, which includes total but also less than total treatment or prevention, and wherein some biologically or medically relevant result is achieved.

The publications discussed above are provided solely for their disclosure before the filing date of the present application. Nothing herein is to be construed as an admission that the present disclosure is not entitled to antedate such disclosure by virtue of prior disclosure.

### EXAMPLES

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1. ANAVEX® 2-73

ANAVEX®2-73 (blarcamesine), an agonist of Sigma-1 receptor (SIGMAR1), has been shown mechanistically focusing on a new target relevant to Alzheimer's disease, Parkinson's disease, Parkinson's disease with dementia, and other neurological diseases. SIGMAR1 activation is a compensatory mechanism to chronic CNS diseases. The direct occupancy of ANAVEX® 2-73 at SIGMAR1 has been established using quantitative PET scan. Full genomic analysis of ANAVEX®2-73-PDD-001 Phase 2 study in patients with Parkinson's Disease with Dementia (PDD) assessed biomarkers of response exploring potential for a Precision Medicine approach. The overall goal of this study was to specify the ANAVEX®2-73 response pathways by identifying the most differentially expressed genes and characterizing the pathways involved.

### Example 2. Study Design of ANAVEX® 2-73 Treating Parkinson's Disease with Dementia

In order to assess the safety, tolerability and efficacy of ANAVEX® 2-73 (blarcamesine) oral capsules in treating cognitive impairment in patients with Parkinson's Disease with Dementia (PDD), a phase 2 study, coded “ANAVEX® 2-73-PDD-001 study” (FIG. 1), was conducted. The study was multi-center, randomized, double blind, placebo-controlled, parallel group with 3-arm. The study included a 2-week Screening/Baseline Observation Period and a 14-week Treatment Period (including a 2 week Titration Period), and a 2-week Safety Follow-Up Period. A total of 132 patients were enrolled in the study and were randomly assigned to three testing arms (FIG. 1). In ANAVEX2-73 High Dose arm, 44 patients were given oral capsules of ANAVEX2-73 once daily in an escalating dose of 10 mg, 20 mg, 30 mg and 50 mg. In ANAVEX2-73 Mid Dose arm, 44 patients were given oral capsules of ANAVEX2-73 once daily in an escalating dose of 10 mg, 20 mg, and 30 mg. In placebo group, 44 patients were given placebo capsules. Outcome measures included Cognitive Drug Research (CDR) Computerized Assessment System on Continuity of Attention; Change from Baseline to End of Treatment in Continuity of Attention as measured by Cognitive Drug Research (CDR) Computerized Assessment System Continuity of Attention; Number of participants with treatment-related adverse events as assessed by CTCAE v4.0 [Time Frame: 14 weeks]; Assessment of the safety and tolerability of ANAVEX2-73 compared to placebo; MDS-UPDRS Part III Total Score (Motor Scores); Change from baseline to End of Treatment as measured by MDS-UPDRS Part III Total Score (Motor Scores); SDS-CL-25 [Time Frame: 14 weeks]; Incidence of sleep disorders symptoms (SDS-CL-25); and DNA/RNA Sequencing. Since MDS-UPDRS is indicative of the motor activity for Parkinson Disease (PD), the trial can also identify the effects of ANAVEX® 2-73 on PD patients. In conclusion, the PDD-001 Phase 2 trial provided an opportunity to identify potential effects of ANAVEX®2-73 on both PD and PDD related endpoints.

The trial enrolled 132 adults, ages 50-85, with PDD, and was successfully concluded in September 2020. Participants, recruited at multiple sites in Spain and Australia, were randomly assigned to one of two doses groups (with an escalating ANAVEX 2-73 dose up to 30 or 50 mg oral capsules) or to a placebo, once daily for 14 weeks (see FIG. 1 for details). The trial's main goals were to assess the therapy's safety and changes in patients' cognitive function, measured using the cognitive drug research computerized assessment system—a test that looks at multiple cognitive skills, such as choice reaction time (choosing between similar objects), vigilance, and episodic memory (remembering new personal experiences or shared information). Secondary goals included changes in patients' motor abilities—assessed with the Movement Disorders Society Unified Parkinson's Disease Rating Scale (MDS-UPDRS) Part III—and sleep quality. Previously reported results showed that ANAVEX 2-73 was generally safe, significantly improved several cognitive skills, and lessened REM sleep behavior disorder, in which patients physically act out while dreaming. This sleep disorder is associated with greater cognitive impairment and a higher risk of dementia. Data obtained from the current trial confirmed these cognitive and sleep benefits, and showed that the therapy's high dose (50 mg) led to statistically significant and clinically meaningful reductions in motor and non-motor Parkinson's symptoms, as assessed with the MDS-UPDRS total score. After 14 weeks, the MDS-UPDRS total score dropped by 10.98 points (indicating an easing in disease severity) in the high-dose group and rose by 3.53 points in the placebo group. This represented an adjusted mean improvement of 14.51 points—surpassing the established, clinically meaningful 7.1 point-cut-off—and a relative improvement of 18.9% over 14 weeks. These findings highlight ANAVEX 2-73's ability to not only slow, but also reverse, symptoms that progress in Parkinson's disease, potentially fulfilling an urgent unmet medical need, Anavex stated in its release. ANAVEX 2-73's use by patients also resulted in a significant increase in SIGMAR1 levels, a rise that was significantly associated with improvements in attention measures, and reductions in both MDS-UPDRS total and MDS-UPDRS Part III (motor scores). Overall, these data confirmed the therapy's mechanism of action and supported SIGMAR1 as a biomarker of clinical response to ANAVEX 2-73. After completing the PDD-001 trial, patients had the option of being treated with ANAVEX 2-73 for nearly one year in its extension study, called ANAVEX 2-73-PDD-EP-001 (NCT04575259).

### Example 3. Patients' Genomic Sequencing and ANAVEX 2-73 Response Pathways

In order to gain insights into ANAVEX 2-73 response pathways, during the Phase 2 study, patients' DNA and RNA sequencing were analyzed. Transcriptomic samples of 65 patients enrolled in either High Dose or Mid Dose arms were collected at baseline and at the end of treatment (Week 14). The expression of 14,150 genes was measured. No transformation was applied to the TPM values. Values associated with a with RIN s 4.0 were discarded. The actual dose of ANAVEX 2-73 (blarcamesine) that was given to each patient during the trial was assessed and integrated into the database. To characterize the gene dysregulations, a pathway database was built using an aggregation of public databases including KEGG, Panther, Allen, Brain Atlas, Gene Ontology, and Reactome.

Specifically, as shown in FIG. 2A, gene analysis was performed as follows: RNA sequencing was done to the collected 130 samples and the expression of 14,150 genes was measured. The genes were clustered together using WGCNA, starting from their correlation of TPM expression values across samples. Seventeen clusters with size ranging from 13 to 11,190 genes were obtained through this gene clusterisation. The TPM expression values of genes in clusters were then aggregated in a single “eigengene” for each cluster. This eigengene served as a representant of the overall dysregulation of the cluster. Secondly, a cluster filtering was performed, wherein patients' arm and dose information were taken into account based on mixed effect models. For each cluster, the eigengene was used as target variable of a linear mixed effect model with 3 covariates: ActualDose, Patient, and timepoint. For each cluster, the contrast ActualDose, i.e. treated vs placebo were evaluated across all timepoints. P-values were obtained with Dunnett's test. Regardless of the cluster and pathway, pathways with less than 4 genes in a cluster were removed, to compensate for the large variability of cluster size and corresponding variability of number of pathways identified in each clusters. The ratio between cluster size and number of pathways was calculated, and top 2 clusters were retained for further Analysis. Cluster 1 contained 213 genes (n1=213) and 37 pathways, and Cluster 2 contained 962 genes (n2=962) and 317 pathways. Both clusters possessed dysregulations significantly linked to the highest dose of ANAVEX 2-73 (blarcamesine). Thirdly, the two retained clusters were characterized and underwent pathway analysis. The characterization and analysis were applied to all the genes of the cluster (whole cluster), and to the genes of the clusters that are known to have biological interactions (reduced cluster). In the whole cluster analysis, overrepresented pathways were identified using EnrichR package with the parameters of Fisher's adjusted p-value p<0.05 (Benjamini-Hochberg adjustment for multiple testing) and minimum pathway size 20. In the reduced cluster analysis, genes that have no interaction with any other genes of the cluster were discarded, resulting in a reduced cluster. Pathway analysis was performed on these reduced clusters. False Discovery Rate with adjustment for multiple testing (Benjamini-Hochberg procedure) was used to characterize pathways' over-representation. Known interactions between the genes of the cluster were assessed using STRING database with the parameters of Edge score >0.150, and no text mining edges. Known interactions were obtained from public databases, such as KEGG, Panther, Allen, Brain Atlas, Gene Ontology, and Reactome.

As shown in FIG. 3, a significant association was identified between the treatment and the estimated marginal mean expression of 6 clusters. Theses means were larger for the significant clusters, which confirmed the impact of ANAVEX® 2-73 on these clusters' expression. For the first cluster, it was discovered that among its 37 pathways, 10 pathways were significantly overrepresented, and six of them were related to neurogenerative diseases. Further, 76% (164 genes out of 213 genes in Cluster 1) of the genes involved cluster 1 were known to have biological interactions. These 164 genes were significantly (FDR(1)=0.003) enriched for Parkinson's disease, Prion disease and Huntington's disease pathways. For Cluster 2, it was discovered that among its 317 pathways, 10 pathways were significantly overrepresented, and six of them were related to neurogenerative diseases. Further, 76% (164 genes out of 213 genes in Cluster 1) of the genes involved cluster 1 were known to have biological interactions. These 164 genes were significantly (FDR(1)=0.003) enriched for Parkinson's disease, Prion disease and Huntington's disease pathways

TABLE 1
Ten Pathways Significantly Overrepresented In The First Cluster
(n1 = 213 genes)

| Term⁽¹⁾ | Overlap⁽²⁾ | Adjusted p-value⁽³⁾ |
|---|---|---|
| Pathways of neurodegeneration | 15/475 | 0.005 |
| Alzheimer disease | 15/369 | 0.001 |
| Amyotrophic lateral sclerosis | 14/364 | 0.001 |
| Huntington disease | 13/306 | 0.001 |
| Prion disease | 13/273 | 0.001 |
| Parkinson disease | 12/249 | 0.001 |
| Diabetic cardiomyopathy | 9/203 | 0.007 |
| Non-alcoholic fatty liver disease | 8/155 | 0.006 |
| Oxidative phosphorylation | 8/133 | 0.003 |
| Proteasome | 4/46 | 0.028 |

⁽¹⁾Pathways database: KEGG 2021;
⁽²⁾Number of genes in the pathway that are also present in the cluster/number of genes in the pathway;
⁽³⁾Fisher's test; adjustment for multiple testing using Benjamini-Hochberg procedure.

For the second cluster, it was discovered that among its 317 pathways, 15 pathways were significantly overrepresented, and six of them were related to neurogenerative diseases (FIG. 5A). Further, 74 brain areas were significantly overrepresented in the second cluster (n2=962 genes). 11 areas out of these 74 were linked to the olfactory bulb, which is known to be a common symptom in PD and AD. Further, 70% (680 genes out of 967 genes in Cluster 2) of the genes involved cluster 1 were known to have biological interactions. These 680 genes were significantly (FDR(1)<0.001) enriched in Parkinson's disease pathways.

TABLE 2
Eleven Brain Areas Significantly Overrepresented In The Second
Cluster (n1 = 962 genes) and Linked to Olfactory Bulb

| | | Adjusted |
|---|---|---|
| Term⁽¹⁾ | Overlap⁽²⁾ | p-value⁽³⁾ |
| Ribosome | 96/158 | <0.001 |
| Coronavirus disease | 80/232 | <0.001 |
| Pathways of neurodegeneration | 50/475 | <0.001 |
| Huntington disease | 48/306 | <0.001 |
| Alzheimer disease | 47/369 | <0.001 |
| Parkinson disease | 46/249 | <0.001 |
| Amyotrophic lateral sclerosis | 46/364 | <0.001 |
| Thermogenesis | 45/232 | <0.001 |
| Prion disease | 45/273 | <0.001 |
| Oxidative phosphorylation | 36/133 | <0.001 |
| Diabetic cardiomyopathy | 36/203 | <0.001 |
| Non-alcoholic fatty liver disease | 34/155 | <0.001 |
| Retrograde endocannabinoid signaling | 24/148 | <0.001 |
| Cardiac muscle contraction | 11/87 | 0.049 |
| OProteasome | 9/46 | 0.005 |

⁽¹⁾Pathways database: KEGG 2021;
⁽²⁾Number of genes in the pathway that are also present in the cluster/number of genes in the pathway;
⁽³⁾Fisher's test; adjustment for multiple testing using Benjamini-Hochberg procedure.

TABLE 3
Fifteen Pathways Significantly Overrepresented In The Second
Cluster (n1 = 962 genes)

| | | Adjusted |
|---|---|---|
| Term⁽¹⁾ | Overlap⁽²⁾ | p-value⁽³⁾ |
| Anterior olfactory nucleus, layer 1 | 36/300 | <0.001 |
| Main olfactory bulb, glomerular layer | 36/300 | <0.001 |
| Anterior olfactory nucleus, external part | 34/300 | <0.001 |
| OB olfactory fiber layer | 33/300 | 0.001 |
| Olfactory bulb, principal part | 32/300 | 0.002 |
| Main olfactory bulb | 31/300 | 0.004 |
| Main olfactory bulb, outer plexiform layer | 30/300 | 0.007 |
| Main olfactory bulb, inner plexiform layer | 28/300 | 0.026 |
| Main olfactory bulb, mitral layer | 28/300 | 0.026 |
| Anterior olfactory nucleus | 27/300 | 0.040 |
| Olfactory areas | 27/300 | 0.040 |

⁽¹⁾Pathways database: Allen Brain Atlas-olfactory terms only;
⁽²⁾Number of genes in the pathway that are also present in the cluster/number of genes in the pathway
⁽³⁾Fisher's test; adjustment for multiple testing using Benjamini-Hochberg procedure

### Example 4. RNAseq Analysis to Identify the Treatment Impact on Compensatory Pathways of PDD

Whole Blood Transcriptomics Analysis (RNAseq) of patients with Parkinson's disease with dementia (PDD) was performed for the ANAVEX®2-73-PDD-001 study (FIG. 1) at two timepoints: Baseline and Week 14 (end of trial). All available RNA samples with RIN≥41 (RNA integrity number, using 130 samples) were analyzed. The expression of 14,150 genes at these two timepoints were analyzed from both placebo and ANAVEX® 2-73 treated patients.

The above method was performed as illustrated in FIG. 2B. In genes clusterization, genes were clustered together using WGCNA1 (Weighted correlation network analysis. Correlation networks are being used in biology to analyze large, high-dimensional data sets), starting from their correlation of TPM2 expression (Transcript per million. TPM value represents a relative expression level that should be comparable between samples) values across samples: WGCNA created 17 clusters (size: 13 to 11,190 genes). Then, the cluster's eigengene3 (one of a set of a genes matrix that tabulates the mRNA or gene expression of the genes across the sample) was used as summary of expression level of the cluster. In cluster filtering, the eigengene was used for each cluster as target variable of a linear mixed effect model with 3 covariates: Dose, patient, and timepoint. Clusters without significant (Dunnett's test) contrasts between treated and placebo across all timepoints were filtered out. The ratio between cluster size and number of pathways was calculated and the top 2 clusters were eventually retained. In clusters biological description, each cluster's functional interactions between the genes of the cluster were assessed using the STRING database (Edge score >0.150; no text mining edges). Genes that have no interaction with any other genes of the cluster were discarded, which led to a reduced cluster. Pathway enrichment analysis was performed on these reduced clusters. False Discovery Rate (FDR, adjustment for multiple testing with the Benjamini-Hochberg procedure) with adjustment for multiple testing (Benjamini-Hochberg procedure) was used to characterize pathway overrepresentation.

Randomized, placebo-controlled clinical trial in 132 patients with Parkinson's disease with dementia (PDD) included prespecified biomarkers of response as well as Whole Exome Sequencing DNA data and full RNA exome expression data collection. This study demonstrated dose-dependent, statistically significant improvement of dementia assessment, Quality of Episodic Memory with ANAVEX® 2-73 (p=0.003) as well as significant improvement of Parkinson's assessment, MDS-UPDRS Total score (p=0.034) for patients treated with ANAVEX® 2-73 high oral dose once daily during 14-week trial.

ANAVEX® 2-73 transcriptomics analysis (RNAseq), identified a gene network that is differentially expressed in Parkinson's disease with dementia (PDD) patients treated with ANAVEX® 2-73 compared to placebo after 14 weeks of treatment. Biological relevance of this gene network was assessed through pathway analysis and confirmed the impact of ANAVEX® 2-73 treatment on pathways involved in neurodegenerative diseases, especially Alzheimer's disease and Parkinson's disease. In the two clusters of genes impacted by ANAVEX® 2-73, over 70% of the genes are known to have biological interactions. Pathway enrichment analysis revealed that multiple neurodegenerative pathways, including Alzheimer's disease and Parkinson's disease were significantly (adjusted p-value <0.005) enriched for these genes, as shown in FIG. 4B and FIG. 5B. Both identified clusters were up-regulated for treated patients compared to placebo: Cluster 2 eigengene expression was significantly increased for patients treated with ANAVEX® 2-73 high oral dose compared to placebo (p=0.021). These genes are known to be down-regulated in the pathology of both Alzheimer's disease and Parkinson's disease. Thus, it is reasonable to conclude that expression levels of dysregulated neurodegenerative genes were restored by the therapeutic effect of ANAVEX® 2-73.

In summary, the study identified a unique set of genes impacted by ANAVEX2-73 (blarcamesine) treatment. The genes were differentially expressed, and their respective expression was correlated to a range of neurodegenerative disorders, such as Alzheimer's disease, Parkinson's disease, Huntington's disease, Amyotrophic lateral sclerosis, Prion disease, diabetic cardiomyopathy, non-alcoholic fatty liver disease, a coronavirus disease, a disorder related to thermogenesis, a disorder related to oxidative phosphorylation, a disorder related to proteasome dysfunction, a disorder related to ribosome dysfunction, a disorder related to retrograde endocannabinoid signaling, a disorder related to cardiac muscle contraction. Pathway analysis confirmed the impact of the treatment on pathways involved in neurodegenerative diseases, as well as the association between ANAVEX2-73 (blarcamesine) and genes differentially expressed across multiple brain areas. Characterization of the ANAVEX2-73 (blarcamesine) response pathway identified the opportunity for translational identification of potential indications for ANAVEX2-73 (blarcamesine), including Alzheimer's disease, Parkinson's disease, Huntington's disease, Amyotrophic lateral sclerosis, Prion disease, diabetic cardiomyopathy, non-alcoholic fatty liver disease, a coronavirus disease, a disorder related to thermogenesis, a disorder related to oxidative phosphorylation, a disorder related to proteasome dysfunction, a disorder related to ribosome dysfunction, a disorder related to retrograde endocannabinoid signaling, a disorder related to cardiac muscle contraction.

These results provide guidance and facilitation on contextualization of upcoming readout of ANAVEX® 2-73 Phase 2b/3 Alzheimer's disease clinical trial. These findings further support pivotal studies in Parkinson's disease and Parkinson's disease with dementia. These findings also enable the translational identification of additional potential therapeutic indications for ANAVEX® 2-73.

Results showed while genes were down-regulated in Alzheimer's disease and Parkinson's disease, ANAVEX®2-73 singularly impacted expression levels of these genes in multiple pathways by countering the pathological down-regulation of genes in both Alzheimer's (p<0.005) and Parkinson's disease (p<0.005) and other degenerative diseases (p<0.005) and these may represent additional potential biomarkers of disease pathology and response.

### Example 5. AI Analysis to Identify New Biomarkers for PD and PDD

As shown in FIG. 6, Parkinson's Disease (PD) is a highly heterogeneous multisystem disorder, some patients would progress to PDD. PD is the second most common neurodegenerative disorder where also cognitive impairment, such as characterized PDD, is common. The PDD-001 Phase 2 study incorporated endpoints related to both PD and PDD (FIG. 7), thus is capable of illustrating the potential effects of ANAVEX® 2-73 on both PD and PDD. In order to gain further insights into ANAVEX 2-73 response pathways against Parkinson disease and PDD, patients' data, including sequence profiles and others, obtained from PDD-001 trial (FIG. 1) were further analyzed by Artificial Intelligence (AI) aided Computer Modeling (AI Modeling) to identify key new biomarkers. As shown in FIG. 8, the AI Modeling considered a wide range of factors and/or variables, include clinical factors, Omics (genomics, proteomics, and phenomics), Actigraphy-sleep data, and interacted them variables such as time-points, doses and end-points. Clinical factors included clinical assessments, patient's co-medications, age, gender, and weight. Omics refers to data on genomics, proteomics, and phenomics. Actigraphy data are sleep parameters and average motor activity over a period of days to weeks using a noninvasive accelerometer, such as one housed in a wristwatch. Time points used were baseline time, Week 8 and Week 14 post-administration. Doses included placebo dose, low, medium, and high doses. End points were clinical assessments based on MDS-UPDRS, genetic variant analysis and COMT as defined in the clinical trial. Through AI Modeling, 24,848,764 relationships were generated. AI Modeling identified three (3) biomarkers for patient selection.

The three biomarkers identified are MS4A4A gene, and its single nucleotide polymorphism variants rs6591561 and rs1582763. These biomarkers displayed confidence* ≥0.75, Lift* ≥1.5, Fisher's p<0.05. Their concordant evolutions were observed with dose and with the subgroup levels improved outcome at respective dose, and effect, at least 2 treatment doses, 2 subgroup levels and 2 different evolutions for the endpoint. It was found that MS4A4A gene mutation functioned as a switch for microglia metabolism which correlated with disease progression in AD, PD and PDD. As shown in FIG. 9, in disease conditions, homeostatic microglia can transition to either a pro-inflammatory (Mic 1) or anti-inflammatory (Mic 3) state. Mic 3 cells are themselves influenced by protective (green) and risk (red) SNPs in the MS4A4A locus, which push these cells into either a helpful, interferon-based response with efficient lipid metabolism (left), or a destructive inflammatory response that accumulates lipids (right).

SNP rs6591561, a variant in the MS4A4A coding region that raises AD risk and was also present in nearly one-third of the cohort. This risk variant altered expression of 1,597 microglial genes, powering proinflammatory proteins such as cytokines, while suppressing interferon signaling and lipid metabolism genes. It lowered expression of MS4A4A itself. Therefore, rs6591561 was identified as a risk variant in MS4A4A.

On the other hand, rs1582763 was found to be a protective variant in the MS4A4A locus, by promoting anti-inflammatory microglia. These microglia express interferons and metabolize lipids well. Different ANAVEX® 2-73 response depending patients' MS4A4A (rs6591561) carrier status in PDD-001 study was observed.

Based on AI Modeling results, All-Corners Studies were designed to utilize these identified biomarkers in selection of patient populations in targeted medicine (FIG. 10). By applying Biomarker Noise Model shown in FIG. 11, patients with reproducible therapeutic effect are separated from the patient population. This strategy would greatly improve clinical trials by targeting the right participant with the right biomarker in the right trial.

### Example 6. PD Pivotal Trial Phase 3

A PD pivotal trial phase 3 was designed, reflecting sub-scores MDS-UPRDS grouped as Part I, II, III and IV. In the trial, sample size analysis was conducted to detect the signal of both efficacy MDS-UPDRS Total score and efficacy MDS-UPDRS individual scores at Week 14. Sample sizes were computed with pwr package on R and are based on observed Cohen's d effects sizes. Sample sizes were adjusted with the variant prevalence in the general population. This sample size may be used to detect the signal for MS4A4A rs6591561 Wild Type (excluding rs6591561 homozygous) cohort assuming the prevalence is similar to the prevalence in the general population (i.e., 70%) as shown in TABLE 4. Most relevant MDS-UPDRS score [Part II] demonstrated strongest response with MS4A4A Wild Type rs6591561 cohort (excluding rs6591561 homozygous mutation carriers). Detailed analysis and correlation of MDS-UPDRS score versus MS4A4A Wild Type rs6591561 cohort are to be conduct. It is likely that this selection gene [MS4A4A rs6591561 Wild Type (excluding rs6591561 homozygous)] might be also beneficial in other CNS indications using ANAVEX2-73.

TABLE 4
MDS-UPDRS Score and Sample Size Analysis

| | [symbol] | | [symbol] | | [symbol] | | [symbol] | |
|---|---|---|---|---|---|---|---|---|
| | [symbol] | [symbol] | [symbol] | [symbol] | [symbol] | [symbol] | [symbol] | [symbol] |
| [symbol] | 13 | 302 | 23 | 43,685 | 22 | 2,739 | 15 | 1,379 |
| [symbol] | 21 | 102,848 | 21 | 1,272 | 27 | 548 | 23 | 122 |
| [symbol] | 18 | 119 | 25 | 405 | 13 | 28 | 16 | 92 |

[symbol]

indicates data missing or illegible when filed

1-64. (canceled)

65. A method of regulating expression of an MS4A4A gene in a subject in need thereof, the method comprising administering a Sigma Sigma-1 receptor agonist to the subject.

66. The method of claim 65, wherein the subject is having or is suspected of having a microglia dysfunction.

67. The method of claim 66, wherein the microglia dysfunction comprises a neurodegenerative disorder.

68. The method of claim 67, wherein the neurodegenerative disorder is selected from Alzheimer's disease, Parkinson's disease, Parkinson's disease with dementia, Huntington's disease, Amyotrophic lateral sclerosis, Prion disease, and any combination thereof.

69. The method of claim 65, wherein the Sigma-1 receptor agonist is selected from ANAVEX2-73 (A2-73), A2-73 free base, ANAVEX 19-144, ANAVEX1-41, AV1066, ANAVEX3-71, PRE-084, Donepezil, Fluvoxamine, Amitriptyline, L-687,384, SA-4503, Dextromethorphan, Dimethyltryptamine, (+)-pentazocine, any crystal forms, enantiomers and pharmaceutically acceptable salts thereof and any combination thereof.

70. The method of claim 69, wherein the Sigma-1 receptor agonist is selected from A2-73 free base, A2-73 amorphous form, A2-73 Crystal Form I, A2-73 Crystal Form II, A2-73 Crystal Form III, (−A2-73 enantiomer, and (+) A2-73 enantiomer.

71. The method of claim 70, wherein the Sigma-1 receptor agonist is administered daily.

72. The method of claim 71, wherein the Sigma-1 receptor agonist is selected from A2-73 free base and A2-73 amorphous form, and wherein the A2-73 is administered daily in an amount of about 10-100 mg for a period of 6 to 14 weeks.

73. The method of claim 71, wherein the Sigma-1 receptor agonist comprises A2-73 in a crystal form or an optical pure form, and wherein the A2-73 is administered once daily in an amount of about 40-60 mg for a period of up to 11 weeks.

74. The method of claim 71, wherein the Sigma-1 receptor agonist comprises A2-73 in a crystal form or an optical pure form, and wherein the A2-73 is administered once daily for a period of 11 weeks in an escalating amount starting at about 10 mg and ending at about 60 mg.

75. A method of evaluating effectiveness of a neurodegenerative therapy in a subject in need thereof, the method comprising the steps of:
(a) obtaining or having obtained a first biological sample of the subject before onset of the neurodegenerative therapy;
(b) providing the neurodegenerative therapy to the subject for a period of therapy time;
(c) obtaining or having obtained a second biological sample of the subject at the end of the therapy time;
(d) determining MS4A4A gene expression level in the first biological sample and in the second biological sample;
(e) identifying the neurodegenerative therapy as
(i) effective for the subject, if the MS4A4A gene expression level in the first biological sample is lower than that in the second biological sample; or
(ii) ineffective for the subject, if the MS4A4A gene expression level in the first biological sample is higher than that in the second biological sample.

76. The method of claim 75, wherein if the neurodegenerative therapy is identified as ineffective in (e), the method further comprises one or more of the following steps:
(i) switching to another neurodegenerative therapy;
(ii) supplementing with another neurodegenerative therapy;
(iii) adjusting dose if the neurodegenerative therapy involves drug therapy; or
iv) switching to a combined neurodegenerative therapy.

77. The method of claim 75, wherein the first biological sample or the second biological sample is each independently comprises a blood serum sample, a blood plasma sample, or a sample suitable for transcriptomic analysis.

78. The method of claim 75, wherein the neurodegenerative therapy is a therapeutic treatment for treating a disease selected from Alzheimer's disease, Parkinson's disease, Parkinson's disease with dementia, Huntington's disease, Amyotrophic lateral sclerosis, Prion disease, and any combination thereof.

79. The method of claim 75, wherein the neurodegenerative therapy comprises a drug therapy selected from ANAVEX2-73 (A2-73), ANAVEX 19-144, ANAVEX1-41, AV1066, ANAVEX3-71, PRE-084, Donepezil, Fluvoxamine, Amitriptyline, L-687,384, SA-4503, Dextromethorphan, Dimethyltryptamine, (+)-pentazocine, and any of their crystal forms, enantiomers and pharmaceutically acceptable salts.

80. The method of claim 79, wherein the neurodegenerative therapy is selected from a drug therapy comprising A2-73 free base, A2-73 amorphous form, A2-73 Crystal Form I, A2-73 Crystal Form II, A2-73 Crystal Form III, (−) A2-73 enantiomer, and (+) A2-73 enantiomer.

81. The method of claim 80, wherein the period of therapy time is up to 14 weeks.

82. (canceled)

83. The method of claim 81, wherein the neurodegenerative therapy is administered daily.

84. The method of claim 83, wherein the neurodegenerative therapy comprises daily administering A2-73 or A2-73 free base in an amount of about 10-100 mg, and for a period of 6 to 14 weeks.

85. The method of claim 83, wherein the neurodegenerative therapy comprises administering a crystal form or an optically pure form of A2-73 once daily.

86. The method of claim 85, wherein the neurodegenerative therapy comprises administering a crystal form of A2-73 once daily in an escalating amount starting at about 10 mg and ending about 50 mg, and for a period up to 14 weeks.

87. The method of claim 85, wherein the neurodegenerative therapy administering an optically pure form of A2-73 once daily in an escalating amount starting at about 10 mg and ending about 60 mg, and for a period up to 14 weeks.

88-122. (canceled)
